(19) United States
(12) Patent Application Publication
(10) Pub. No.: US 2012/0199005 A1
(43) Pub. Date: Aug. 9, 2012

(54) LIGHTING AND AIR CLEANING DEVICE

(75) Inventors: Masashi Koji, Tokyo (JP); Yuya Koji, Tokyo (JP)

(73) Assignee: HOSHIN KAGAKU SANGYOSHO CO., LTD., Tokyo (JP)

(21) Appl. No.: 13/501,127

(22) PCT Filed: Oct. 18, 2010
(86) PCT No.: PCT/JP2010/068282

§ 371 Date: Apr. 10, 2012

(30) Foreign Application Priority Data
Oct. 19, 2009 (JP) 2009-240734

(51) Int. Cl. A61L 9/20

(52) U.S. Cl. 96/224

(57) ABSTRACT

An ultraviolet lamp, and an ultraviolet-shielding member that covers at least a part of an ultraviolet emission portion are provided. The ultraviolet-shielding member includes an ultraviolet non-transmissive and visible light transmissive portion that blocks or absorbs ultraviolet rays. Further, an air passage in which air around the ultraviolet emission portion is provided between the ultraviolet lamp and the ultraviolet-shielding member.

### TECHNICAL FIELD

The present invention relates to a lighting and air cleaning device that emits an electromagnetic wave from an electromagnetic wave generation source to sterilize bacteria, viruses, and the like floating in the air by utilizing ultraviolet rays in a wavelength region of 180 to 379 nm (nanometers) within the electromagnetic wave (mainly, sterilization rays having a wavelength of 253.7 mm), blocks or absorb the ultraviolet rays for a human, or excites a light emitting substance, and irradiates visible light rays in a wavelength region of 380 to 780 nm, which are present within the electromagnetic wave or which have been generated by exciting the light-emitting material with the ultraviolet rays, to utilize the visible light rays as part of illumination light.

### BACKGROUND ART

In general, as lightings in homes, offices, and the like, fluorescent lamps (fluorescent tubes) are used, and sterilization lamps (ultraviolet sterilization tubes) are used, for example, for killing or sterilizing bacteria in water, food, and the like. The sterilization lamps and the fluorescent lamps are lamps that utilize low-pressure mercury vapor discharge, and basically have substantially the same operation principle and light emission principle. They mainly differ in constituent materials, and in the sterilization lamp, a quartz glass tube or an ultraviolet transparent glass tube which transmits light having a wavelength of 290 nm or less is used, and in the fluorescent lamp, a soda lime glass tube or another glass tube in which a fluorescent member is applied to its inner surface and which blocks ultraviolet rays having a wavelength of 290 nm or less is used.

In the sterilization lamp, when current is caused to flow through an electrode at the time of lighting, thermal electrons are emitted from the electrode, these thermal electrons are attracted by the opposite electrode to be moved, and discharge is started. Electrons that flow by this discharge collide with mercury atoms, and energy released when energy excited by this collision is returned to the original state is referred to as ultraviolet rays. The sterilization rays released here produce the sterilization effect of sterilizing bacteria, viruses, and the like, and visible light rays (380 to 780 nm) released simultaneously illuminate the surrounding area, with the result that illumination effects are produced.

On the other hand, although the light emission principle of the fluorescent lamp is the same as that of the sterilization lamp, they differ in that ultraviolet rays generated within the tube excite the fluorescent member applied on the inner surface of the tube. Specifically, the ultraviolet rays generated within the tube and having a wavelength of 290 nm or less are absorbed by the soda lime glass tube, and excite the fluorescent member, and thus visible light rays are emitted. The emitted visible light rays illuminate the surrounding area, and remarkably significant illumination effects are produced.

It is known that, in general, an electromagnetic wave (ultraviolet rays) within a wavelength region of 180 to 379 nm, in particular, an electromagnetic wave (sterilization rays) having a wavelength of 253.7 nm, has the effect of killing bacteria, viruses, and the like, and sterilizing them. Hence, by using the sterilization lamp that emits ultraviolet rays as an illumination lamp, it is theoretically possible to kill or sterilize bacteria, viruses, and the like floating in the air in the surrounding area, and to simultaneously produce the illumination effect of the fluorescent lamp to illuminate the surrounding area.

However, it is also known that ultraviolet rays are harmful to a human. Specifically, when a predetermined amount or more of ultraviolet rays is irradiated to the skin or the eyes of a human, for example, problems occur in that the skin festers, skin cancer is caused in the long term, and inflammation is caused in the eyes. Furthermore, ultraviolet rays adversely affect common engineering plastics (for example, ABS, PC and POM) either directly or indirectly, degradation of material is accelerated at a part to which ultraviolet rays are irradiated, and thereby small cracks are generated, causing the part to be easily broken and torn, and degrading the durability.

Hence, a conventional sterilization lamp is used only for killing or sterilizing bacteria, viruses, and the like, and is not used as the fluorescent lamp for illumination. For example, such consideration is given that in a place where a human is present or passes, only when a human is not present in the place, the sterilization lamp is turned on to emit ultraviolet rays to kill or sterilize bacteria, viruses, and the like floating in the air in the surrounding area, whereas, when a human is present, the sterilization lamp is turned off so that the ultraviolet rays are not irradiated to the human. Moreover, when the sterilization lamp is constantly turned on in a place where a human is present, the sides of the sterilization lamp facing the human are all shielded, and thus ultraviolet rays are prevented from being irradiated directly to the human. In these cases, the device incorporating the sterilization lamp is a sterilization device having an ultraviolet sterilization lamp, and cannot be said to be an illumination device for illuminating the surrounding area brightly for a human.

Furthermore, given that when glass which does not transmit ultraviolet rays having a wavelength region of 180 to 379 nm is attached to a side facing a human, the glass is likely to be broken or damaged, there is a possibility that, for example, the broken glass disadvantageously causes injuries. In case of a disaster such as an earthquake or a typhoon, pieces of glass become dangerous, and the pieces of glass are likely to cause a large problem.
Patent document 1: Japanese Unexamined Patent Application Publication No. 2000-167035
Patent document 2: Japanese Unexamined Patent Application Publication No. 2000-228112
Patent document 3: Japanese Unexamined Patent Application Publication No. 2000-182432
Patent document 4: Japanese Unexamined Patent Application Publication No. 2000-207903
Patent document 5: Japanese Unexamined Patent Application Publication No. 2006-181000

As the first example of a conventional lighting and air cleaning device, for example, there is the one disclosed in patent document 1. Patent document 1 discloses an air cleaning device that can perform room illumination and air cleaning by itself. The air cleaning device disclosed in patent document 1 includes: a casing that has an air inlet port, an air outlet port, and a visible light ray emission portion; a photocatalyst attached adsorption member that is disposed within the casing; a blower that generates an air current within the casing; and a mercury lamp in which a fluorescent member is formed in a predetermined light distribution range on the valve inner surface. The air cleaning device emits visible light rays into a room, through the visible light ray emission portion, from a valve external surface corresponding to the part of the mercury lamp where the fluorescent member is formed, and emits ultraviolet rays to the photocatalyst attached adsorption member from a valve external surface corresponding to the part where the fluorescent member is not formed.

According to this air cleaning device (hereinafter referred to as the “first conventional example”), an effect is expected that visible light rays are emitted inside the room, through the visible light ray emission portion of the casing, from the valve external surface corresponding to the part where the fluorescent member is formed, and thus the room is illuminated and is not dark.

As the second example of the conventional lighting and air cleaning device, there is also the one disclosed in patent document 2. Patent document 2 discloses an illumination device in which an air cleaning function is provided to an illumination unit. The illumination device disclosed in patent document 2 is characterized in that an air cleaning device having a photocatalyst to which ultraviolet rays are irradiated from an ultraviolet lamp, an air filter, and a fan which forcibly circulates air to the air filter from a predetermined direction is provided in the illumination unit having an illumination light source and lighting means thereof.

According to this illumination device (hereinafter referred to as the “second conventional example”), an effect is expected that the air around the illumination unit is cleaned by the photocatalyst type air cleaning device provided in the illumination unit, thereby the stain of the illumination unit is reduced, and the illumination efficiency of the illumination unit is kept high for a long period of time.

As the third example of the conventional lighting and air cleaning device, there is also the one disclosed in patent document 3. Patent document 3 discloses a sterilization tube unit. In the sterilization tube unit disclosed in patent document 3, a duct is provided within the body of the unit, a blower fan and a sterilization tube lamp are arranged within the duct, the sterilization tube lamp irradiates ultraviolet rays to the air sucked through an air inlet port provided at one end of the duct, and the sterilization air is blown into a room through an air outlet port provided at the other end of the duct. Heat of the stabilizer of the sterilization tube lamp is dissipated by an air current within the duct.

According to this sterilization tube unit (hereinafter referred to as the “third conventional example”), an effect is expected that, for example, it is possible to achieve much more effective dissipation of heat compared with the dissipation of heat by natural convection such as the dissipation of the heat of the stabilizer from the body of the unit.

As the fourth example of the conventional lighting and air cleaning device, there is the one disclosed in patent document 4. Patent document 4 discloses an illumination device in which an air cleaning function is provided to an illumination unit. The illumination device disclosed in patent document 4 includes: a fluorescent lamp in which a light emission layer having an aperture portion is formed on the inner surface of an ultraviolet transmissive glass valve; and a unit body which directly Or indirectly supports the fluorescent lamp. The fluorescent lamp is supported by the unit body such that the aperture portion is on the side of an attachment surface such as a ceiling surface, and illumination light is emitted from the light emission layer of the fluorescent lamp, and ultraviolet rays are emitted from the aperture portion.

According to this illumination device (hereinafter referred to as the “fourth conventional example”), it is unnecessary to use both a unit for illumination and a sterilization tube unit as conventionally required, and thus an effect is expected that, for example, it is possible to reduce facility cost.

As the fifth example of the conventional lighting and air cleaning device, there is also the one disclosed in patent document 5. Patent document 5 discloses an air cleaning device that can perform deodorization, sterilization, illumination, and the like. The air cleaning device disclosed in patent document 5 includes: a discharge tube which emits ultraviolet light; an illumination portion which converts the ultraviolet light emitted from the discharge tube into visible light and releases the visible light; and a reflective portion in which a photocatalyst is provided in an area where the ultraviolet light emitted from the discharge tube is received. An air current passage is provided between the discharge tube and the reflective portion. The illumination portion has an illumination cover that is made of quartz glass and formed so as to be convexly curved, and a coating of a fluorescent substance is provided on a surface on the side of the discharge tube that is the inside thereof. On the surface on the side of the discharge tube that is the inside of the reflective portion positioned on the opposite side to the illumination portion, a coating of the photocatalyst is provided. A current passage for circulating air is formed between the coating of the fluorescent substance of the illumination portion and the coating of the photocatalyst of the reflective portion.

According to this air cleaning device (hereinafter referred to as the “fifth conventional example”), it is possible to kill, with the ultraviolet light emitted from the discharge tube, bacteria contained in the air within the air current passage, and to decompose organic materials and organic compounds contained in the air within the air current passage by the chemical reaction of the photocatalyst caused by the irradiation of the ultraviolet light from the discharge tube. Also, it is possible to release visible light rays by exciting a light-emitting material (such as a fluorescent substance) utilizing the ultraviolet rays emitted from the discharge tube, and utilize the visible light rays as illumination light.

However, in any of the first conventional example (the air cleaning device), the second conventional example (the illumination device), the fourth conventional example (the illumination device), and the fifth conventional example (part of the example), a fluorescent film is provided only on part of a bulb inner surface of an ultraviolet lamp, and it is constituted such that a fluorescent film formation portion where the fluorescent film is formed and a fluorescent film non-formation portion where the fluorescent film is not formed are provided (hereinafter referred to as an “aperture type fluorescent lamp”). In the aperture type fluorescent lamp, part of the ultraviolet rays emitted by the lamp are made to pass through the fluorescent film formation portion and are emitted as visible light rays, and the rest of the ultraviolet rays is made to pass through the fluorescent film non-formation portion and is directly emitted as the ultraviolet rays.

According to the aperture type fluorescent lamp, since the fluorescent film formation portion and the fluorescent film non-formation portion are present, and thus the emission range of the ultraviolet rays emitted to the outside of the lamp is narrowed, the total amount of emitted ultraviolet rays is reduced, and there has been a problem that the lamp is brought into the same state where the function of the ultraviolet lamp is degraded. Also, in the fluorescent film in the fluorescent film formation portion, since both its ends are not continuous, when the ultraviolet lamp is turned on, the inside thereof being in a vacuum state, the temperature becomes relatively high, and the fluorescent film easily comes off by being affected by the high temperature and electron beams. Hence, when the fluorescent film comes off due to long term use or the like, and the fluorescent member that has come off is attached to the fluorescent film non-formation portion, problems occur in that the emission of the ultraviolet rays is reduced and in addition, the output of the ultraviolet rays is further reduced. Furthermore, since the aperture type fluorescent lamp has a special structure that the fluorescent member is formed only in the predetermined light distribution range on the bulb inner surface, a commercially available ultraviolet lamp cannot be used, and thus there has been a problem that the cost of the entire device is increased.

In the third conventional example (the sterilization tube unit), the duct is provided within the body of the unit, the blower fan and the sterilization tube lamp are arranged within the duct, the sterilization tube lamp irradiates ultraviolet rays to the air sucked through the air inlet port provided at one end of the duct, and the sterilized air is blown through the air outlet port provided at the other end. Hence, even though the sterilization tube unit can dissipate heat of the stabilizer of the sterilization tube lamp, consideration is not given to the use as an illumination device, so that the sterilization tube unit belongs to the concept of a sterilization device using ultraviolet rays.

In the fifth conventional example (the air cleaning device) (an example shown in FIG. 2 of patent document 5), the discharge tube, the illumination portion, and the reflective portion are provided, the air current passage is formed between the surface on the side of the coating of the fluorescent substance of the illumination portion and the surface on the side of the coating of the photocatalyst of the reflective portion, and the discharge tube is disposed within the air current passage. The illumination portion is made of quartz glass and formed so as to be convexly curved, and the coating of the fluorescent substance is provided on the surface on the side of the discharge tube that is the inside of the illumination portion. On the surface on the side of the discharge tube that is the inside of the reflective portion positioned on the opposite side to the illumination portion, the coating of the photocatalyst is provided. The photocatalyst of the reflective portion is made to communicate with outside air through the air current passage. Hence, the emission of ultraviolet rays causes oxidation decomposition in the coating of photocatalyst, and energy of an electromagnetic wave emitted from the ultraviolet lamp is partially left as heat within the quartz glass, and the heat and the energy of the oxidation decomposition generated from the photocatalyst cause the coating of the fluorescent substance adhering to the surface of the quartz glass to be broken and separated, with the result that small pieces of the separated fluorescent substance float in the air. Consequently, not only the fluorescent substance is separated from the illumination portion, and the separated pieces of the fluorescent substance are scattered around the surrounding area of the illumination device, which is undesirable in terms of sanitation, the ultraviolet rays themselves which are harmful to a human body are emitted from the illumination portion, and there is a fear that the air cleaning device will not be able to achieve the function as an illumination device.

### DISCLOSURE OF THE INVENTION

The present invention has been made in view of the above-described conventional problems, and an object of the present invention is to provide a lighting and air cleaning device which utilizes the original properties of ultraviolet rays to kill or sterilize, with ultraviolet rays, bacteria, viruses, and the like floating in the air in the surrounding area, thereby functioning as an ultraviolet sterilization device, and which, at the same time, can function as an illumination unit by illuminating the surrounding area with visible light rays, while cutting ultraviolet rays, the direct irradiation of which to a human will become problematic. The lighting and air cleaning device also cuts electromagnetic waves having wavelengths in the ultraviolet region so as not to be harmful to a human, and can exert a stable function as an illumination unit as well, without scattering dangerous materials, such as separated pieces of a light-emitting material (such as a fluorescent member or a pigment) or pieces of quartz glass, to cause damages to a human.

To overcome the above problems and the like and achieve the above-described object, a lighting and air cleaning device of the present invention is provided with: an electromagnetic wave generation source that emits an electromagnetic wave including an ultraviolet ray and a visible light ray; and an ultraviolet-shielding resin member that covers at least a part of an electromagnetic wave emission portion emitting the electromagnetic wave of the electromagnetic wave generation source so as to prevent the ultraviolet ray included in the electromagnetic wave from being irradiated directly to a human. The ultraviolet-shielding resin member includes an ultraviolet non-transmissive and visible-light transmissive portion that blocks or absorbs the ultraviolet ray emitted from the electromagnetic wave generation source and that transmits the visible light ray emitted from the electromagnetic wave generation source, and an air passage in which air around the electromagnetic wave emission portion can move is provided between the electromagnetic wave generation source and the ultraviolet-shielding resin member. Then, the ultraviolet ray emitted from the electromagnetic wave generation source is irradiated to the air moving in the air passage so as to kill or sterilize bacteria, viruses, and the like in the air, and the visible light ray emitted from the electromagnetic wave generation source and passing through the ultraviolet non-transmissive and visible-light transmissive portion is used as illumination light.

With the configuration described above, a light and air cleaning device according to the present invention can kill or sterilize, with ultraviolet rays, bacteria, viruses, and the like floating in the air, thus function as an air cleaning device, and also can cut ultraviolet rays and transmit only visible light rays, or increase visible light rays by changing the ultraviolet rays into visible light rays through excitation of a light-emitting material (such as a fluorescent member or a pigment) with ultraviolet rays, and irradiate the surrounding area with the visible light rays, thus exhibiting the function as an illumination device. In this way, it is possible to provide a lighting and air cleaning device that can exert both the function of an air cleaning device which cuts ultraviolet rays and is safe and not harmful to a human and the function of an illumination device which emits a large amount of visible light rays and which thereby can emit stable illumination light.

### BRIEF DESCRIPTION OF THE DRAWINGS

FIG. 1 is a perspective view showing a first embodiment of a lighting and air cleaning device according to the present invention;

FIG. 2 is an exploded perspective view of the lighting and air cleaning device shown in FIG. 1;

FIG. 3 is a perspective view showing an embodiment of an ultraviolet-shielding resin member used in the lighting and air cleaning device shown in FIG. 1;

FIG. 4 is a graph showing a relationship between the wavelength and the transmittance of light on FEP and PFA resins as examples of the ultraviolet-shielding resin member used in the lighting and air cleaning device shown in FIG. 1;

FIG. 5 is a graph showing an enlarged main portion A of the transmittance of light on the FEP and PFA resins shown in FIG. 4;

FIG. 6 is an explanatory diagram for explaining the function of the lighting and air cleaning device shown in FIG. 1;

FIG. 7 is an exploded diagram showing an exploded second embodiment of the lighting and air cleaning device of the present invention;

FIG. 8 is an exploded diagram showing an exploded third embodiment of the lighting and air cleaning device of the present invention;

FIG. 9 is an exploded diagram showing an exploded fourth embodiment of the lighting and air cleaning device of the present invention;

FIG. 10 is a perspective view showing the overall configuration of the lighting and air cleaning device shown in FIG. 9;

FIG. 11 is a cross-sectional view of screwed portions of the lighting and air cleaning device shown in FIG. 9;

FIG. 12 is an exploded diagram showing an exploded first variation of the lighting and air cleaning device shown in FIG. 9;

FIG. 13 is an exploded diagram showing an exploded main portion of the lighting and air cleaning device shown in FIG. 12;

FIG. 14 is a diagram of screwed portions of a second variation of the lighting and air cleaning device shown in FIG. 9;

FIG. 15 is a diagram of screwed portions of a third variation of the lighting and air cleaning device shown in FIG. 9;

FIG. 16 is a cross section of an upper portion of a fifth embodiment of the lighting and air cleaning device according to the present invention;

FIG. 17 is a cross-sectional view taken along line V-V of the lighting and air cleaning device shown in FIG. 16;

FIG. 18 is an exploded diagram showing an exploded sixth embodiment of the lighting and air cleaning device of the present invention;

FIG. 19 is a front view of the lighting and air cleaning device shown in FIG. 18;

FIG. 20 is a diagram showing a seventh embodiment of the lighting and air cleaning device according to the present invention;

FIG. 21 is an enlarged cross-sectional view taken along line W-W of the lighting and air cleaning device shown in FIG. 20;

FIG. 22 is an exploded diagram showing an exploded first variation of the lighting and air cleaning device shown in FIG. 20;

FIG. 23 is a cross-sectional view of the same portion as in FIG. 21 of the lighting and air cleaning device shown in FIG. 22;

FIG. 24 is a cross-sectional view of the same portion as in FIG. 21 showing a second variation of the lighting and air cleaning device shown in FIG. 20;

FIG. 25 is a cross-sectional view of the same portion as in FIG. 21 showing a third variation of the lighting and air cleaning device shown in FIG. 20;

FIG. 26 is a diagram showing a fourth variation of the lighting and air cleaning device shown in FIG. 20;

FIG. 27 is a side view of the lighting and air cleaning device shown in FIG. 26;

FIG. 28 is an exploded perspective view showing an eight embodiment of the lighting and air cleaning device according to the present invention;

FIG. 29 is a vertical cross-sectional view of a center portion of the lighting and air cleaning device shown in FIG. 28;

FIG. 30 is a cross-sectional view taken along line X-X of FIG. 26 in the lighting and air cleaning device shown in FIG. 28;

FIG. 31 is an exploded perspective view showing the lighting and air cleaning device shown in FIG. 30;

FIG. 32 is a vertical cross-sectional view of a center portion of a ninth embodiment of the lighting and air cleaning device according to the present invention;

FIG. 33 is a vertical cross-sectional view of a center portion showing a first variation of the lighting and air cleaning device shown in FIG. 32;

FIG. 34 is a vertical cross-sectional view of a center portion of a tenth embodiment of the lighting and air cleaning device according to the present invention;

FIG. 35 is a perspective view showing a cover body of the lighting and air cleaning device shown in FIG. 34;

FIG. 36 is a partially-exploded perspective view of an eleventh embodiment of the lighting and air cleaning device according to the present invention;

FIG. 37 is a cross-sectional view taken along line Y-Y of the lighting and air cleaning device shown in FIG. 36;

FIG. 38 is an exploded perspective view showing a first variation of the lighting and air cleaning device shown in FIG. 36;

FIG. 39 is a perspective view showing a twelfth embodiment of the lighting and air cleaning device according to the present invention; and

FIG. 40 is a cross-sectional view taken along line Z-Z of the lighting and air cleaning device shown in FIG. 37.

### DESCRIPTION OF PREFERRED EMBODIMENTS

A lighting and air cleaning device is realized using a simple structure, which kills or sterilizes bacteria, viruses, and the like floating in the air with ultraviolet rays generated from an electromagnetic wave generation source to thereby function as an ultraviolet sterilization device, and which irradiates the surrounding area with visible light rays generated from the electromagnetic wave generation source and visible light rays converted from the ultraviolet rays to thereby function as an illumination device without being harmful to a human.

### First Embodiment

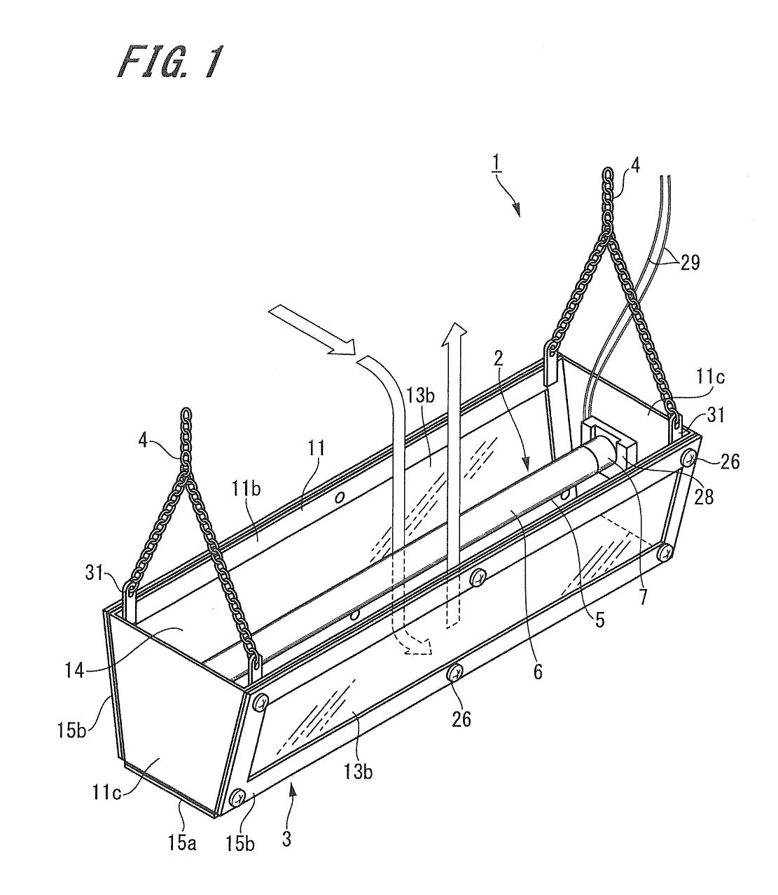

Embodiments of a lighting and air cleaning device according to the present invention will be described below with reference to the accompanying drawings. FIGS. 1 to 6 show the first embodiment of the lighting and air cleaning device according to the present invention. A lighting and air cleaning device 1 described as the first embodiment includes an electromagnetic wave generation source 2 that emits electromagnetic waves including ultraviolet rays, an ultraviolet-shielding resin member 3 that houses the electromagnetic wave generation source 2, and a hanging unit 4.

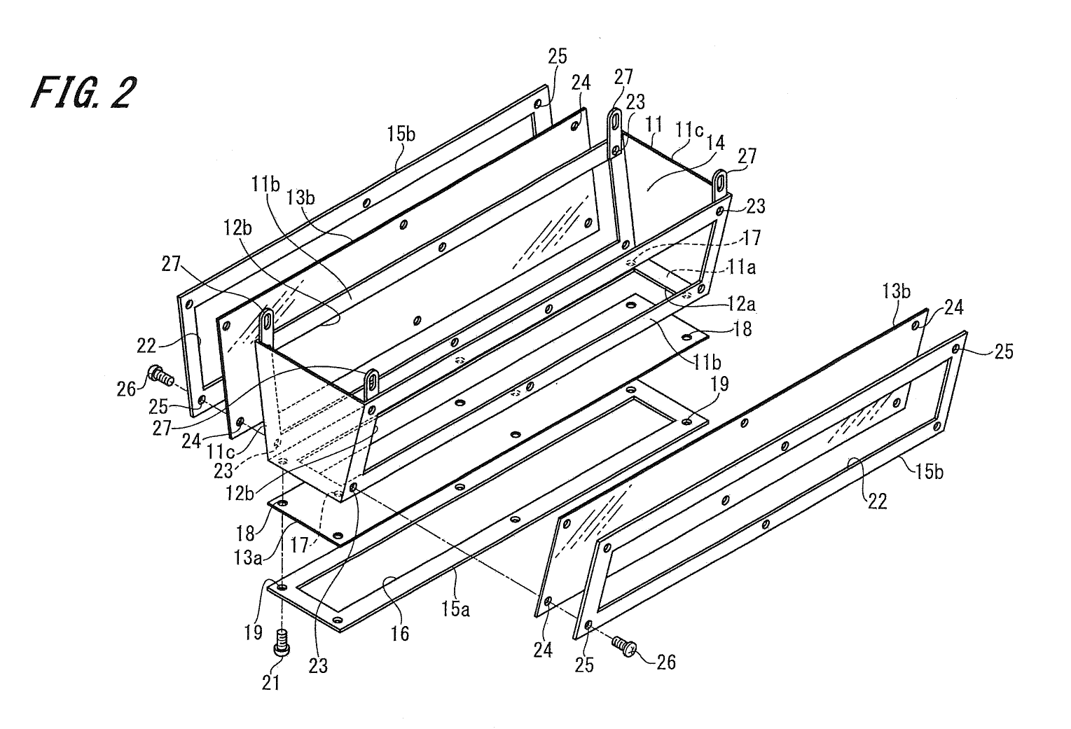

The electromagnetic wave generation source 2 emits electromagnetic waves including an electromagnetic wave (ultraviolet rays) in a wavelength range of 180 (nanometers) to 379 nm and an electromagnetic wave (visible light rays) in a wavelength range of 380 to 780 nm. Examples of this electromagnetic wave generation source 2 can include an ultraviolet lamp, and an ultraviolet generation LED, a semiconductor light source, etc. which will be described later. In the embodiment shown in FIGS. 1 and 2, as the electromagnetic wave generation source 2 of the lighting and air cleaning device 1, a commercially available common ultraviolet lamp 5 is used.

The ultraviolet lamp 5 includes a glass tube 6 that is formed of quartz glass or ultraviolet transparent glass in the shape of a bar, and a pair of ferrules 7, 7 that is formed integrally so as to close both ends of the glass tube 6. Within the ferrule 7, an electrode is held by stem glass, and a pair of ferrule pins 8, 8 that protrudes to the outside from an end surface on the opposite side is connected to both ends of the electrode such that energization can be achieved. Further, mercury and argon gas and the like are sealed in the glass tube 6, and a discharge tube is configured as a whole.

This ultraviolet lamp 5 emits electromagnetic waves including ultraviolet rays and visible light rays. A common output of the ultraviolet lamp 5 is such that ultraviolet rays are about 15 to 30%, visible light rays are about 50 to 60%, and the rest is consumed as heat energy. In the output described above, ultraviolet rays in a wavelength range of 180 to 379 nm have a strong line spectrum (a sterilization ray), in particular at a wavelength of 253.7 nm.

In the ultraviolet lamp 5 described above, when a current is made to flow through the electrode at the time of lighting, thermal electrons are discharged from a filament, the thermal electrons are attracted by and moved to the opposite electrode, and discharge is started. The electrons that are made to flow by this discharge collide with mercury atoms, energy that is discharged when energy excited by this collision is returned to the original state is ultraviolet rays, and the ultraviolet rays pass through the glass tube 6 and are emitted to the outside of the glass tube 6. At the same time, visible light rays also pass through the glass tube 6, and are emitted to the outside of the glass tube 6. The portion of the glass tube 6 that emits the ultraviolet rays and the visible light rays is an ultraviolet emission portion.

The ultraviolet-shielding resin member 3 covers at least part of the ultraviolet emission portion of the ultraviolet lamp 5, and thereby prevents the ultraviolet rays from being directly irradiated to a human. As shown in FIGS. 1 and 2, the ultraviolet-shielding resin member 3 according to the first embodiment is formed of: a casing 11 which houses the ultraviolet lamp 5; an ultraviolet non-transmissive and visible-light transmissive resin plate or an ultraviolet non-transmissive and visible-light transmissive resin film (hereinafter collectively referred to as an “ultraviolet-shielding plate”) 13 that is attached to an opening window 12 provided in the casing 11; and the like.

The casing 11 is formed of a horizontally long enclosure having an opening portion 14 in an upper surface. The casing 11 has a horizontally long lower surface portion 11a, a pair of first side surface portions 11b, 11b that rises continuously on both sides of the lower surface portion 11a in the width direction, and a pair of second side surface portions 11c, 11c that rises continuously on both sides of the lower surface portion 11a in the longitudinal direction. The pair of second side surface portions 11c, 11c is formed in an inverted trapezoid, with the length of a side on the lower surface portion 11a side made longer than a side on the opening portion 14 side, and thus the area of the lower surface portion 11a is greater than that of the opening portion 14. The sides where the lower surface portion 11a is in contact with the first and second side surface portions 11b and 11c are joined, respectively, and thus the surface portions 11a, 11b, and 11c are integrally formed.

Furthermore, in the lower surface portion 11a, edge portions with appropriate width are left at the four sides, and thus a lower surface opening window 12a that is widely opened to form a rectangular shape is provided. Moreover, in each of the pair of first side surface portions 11b, 11b, edge portions with appropriate width are left at the four sides, and thus a side surface opening window 12b that is widely opened to form a rectangular shape is provided. A first ultraviolet-shielding plate 13a that blocks or absorbs ultraviolet rays and that transmits visible light rays is attached to the lower surface opening window 12a; and a second ultraviolet-shielding plate 13b that blocks or absorbs ultraviolet rays and that transmits visible light rays is likewise attached to each of the pair of the side surface opening windows 12b, 12b.

The necessary properties of these ultraviolet-shielding plates 13a and 13b are to block or absorb ultraviolet rays, to be appropriately durable against ultraviolet rays, and to transmit visible light rays. Examples of the material that satisfies these properties include the following materials.

The first one is a single ultraviolet transmissive fluorine resin, and it is formed of only an ultraviolet transmissive and visible-light transmissive fluorine resin. This single ultraviolet transmissive and visible-light transmissive fluorine resin can be said to be the one for considering the thickness of the fluorine resin, and can be formed by setting the thickness of the plate of ultraviolet transmissive and visible-light transmissive fluorine resin to 5 mm or more. In general, fluorine resin is a little whitish but is substantially colorless and transparent, transmits ultraviolet rays relatively easily, and has high durability against ultraviolet rays. However, when the thickness of the plate is 2 mm, the ultraviolet transmittance of the fluorine resin is reduced to about 25%. When the thickness of the plate of the fluorine resin is 4 mm, the ultraviolet transmittance is greatly reduced to 1 to 2%; when the thickness of the plate is 5 mm, most of ultraviolet rays are absorbed, and the transmittance is close to 0%. Hence, by making a plate material having a thickness of 5 mm or more with the ultraviolet transmissive and visible-light transmissive fluorine resin, it is possible to form an ultraviolet-shielding resin member only with the ultraviolet transmissive and visible-light transmissive fluorine resin.

However, fluorine resin is poor in processibility, and the cost is increased as the weight of the ultraviolet transmissive and visible-light transmissive fluorine resin is increased. Also, it is disadvantageous in that it is difficult to manufacture the ultraviolet-shielding resin member with the ultraviolet transmissive and visible-light transmissive fluorine resin having a desired dimensional shape, and large amounts of steps and cost are uneconomically needed. However, when it is possible to manufacture the ultraviolet-shielding resin member only with the ultraviolet transmissive and visible-light transmissive fluorine resin, regardless of the disadvantage described above, because a coating or the like for acquiring non-transmissiveness of ultraviolet rays is not needed, it is possible to enhance productivity in this point. Note that even the fluorine resin having a thickness of 5 mm or more can easily transmit visible light rays.

The second one can be said to be a mixture fluorine resin. A color fluorine resin is obtained by mixing a pigment (a material that contains coloring matter of white, blue, yellow or the like and is used for coloring an object) or a fluorescent member with fluorine resin, and is formed into a plate material or a film material as the color fluorine resin. Since, in general, fluorine resin is colorless and transparent, a light-emitting material such as a pigment or fluorescent member of an appropriate color is added to and mixed with the fluorine resin, and they are integrally formed into a plate or film material, and thus it is possible to transmit visible light rays, and to block or absorb ultraviolet rays, while converting part of the ultraviolet rays into visible light rays through excitation of the light-emitting material such as a pigment or a fluorescent member. Therefore, by containing the light-emitting material (such as a pigment or a fluorescent member) in the fluorine resin and forming them as an integrated plate or film material, it is possible to block or absorb ultraviolet rays and transmit only visible light rays.

In the fluorine resin plate material and the fluorine resin film material each containing the light-emitting material, when an electromagnetic wave generation source is disposed on one surface side, and an electromagnetic wave is emitted therefrom, visible light rays included in the electromagnetic wave pass through the fluorine resin plate material or the fluorine resin film material, and are released from the other surface. By contrast, part of ultraviolet rays included in the emitted electromagnetic wave is converted into visible light rays through excitation of the light-emitting material, and is released from the other surface. Since the rest of the ultraviolet rays is blocked or absorbed by the light-emitting material contained in the fluorine resin plate material or the fluorine resin film material, ultraviolet rays are not released from the other surface.

The third one can be said to be an ultraviolet transmissive composite fluorine resin, in which a coating layer is formed on one surface of an ultraviolet transmissive and visible-light transmissive fluorine resin plate material or an ultraviolet transmissive and visible-light transmissive fluorine resin film member by coating a light-emitting material. This ultraviolet transmissive composite fluorine resin can be divided into a first composite fluorine resin in which the coating layer is provided on a surface facing an electromagnetic wave generation source, and a second composite fluorine resin in which the coating layer is provided on the surface opposite to the electromagnetic wave generation source. The coating layers may be provided on both surfaces of the ultraviolet transmissive and visible-light transmissive fluorine resin. Here, in the present invention, the “coating” is assumed to include spraying, immersion, spreading, wrapping, adhering, and other coating means.

In any of the first and second ultraviolet transmissive composite fluorine resins of the stacking structure of the third one, when an electromagnetic wave is emitted with the surface of the coating layer facing the electromagnetic wave generation source, visible light rays included in the electromagnetic wave directly transmit the ultraviolet transmissive and visible-light transmissive fluorine resin plate material or the ultraviolet transmissive and visible-light transmissive fluorine resin film member and are released from the other surface. By contrast, in the first ultraviolet transmissive composite fluorine resin, the ultraviolet rays included in the emitted electromagnetic wave excite, before transmitting the ultraviolet transmissive and visible-light transmissive fluorine resin, the light-emitting material (such as a pigment or a fluorescent member) coating the front surface of the ultraviolet transmissive and visible-light transmissive fluorine resin, and disappear, and visible light rays generated instead transmit, together with the visible light rays directly passing, the ultraviolet transmissive and visible-light transmissive fluorine resin, and are released from the other surface to the outside.

On the other hand, in the second composite fluorine resin, part of ultraviolet rays transmit the ultraviolet transmissive and visible-light transmissive fluorine resin, and excite the light-emitting material coating the rear surface of the ultraviolet transmissive and visible-light transmissive fluorine resin, and disappear, and visible light rays generated instead, together with the visible light rays directly passing, are released to the outside. Hence, in the first and second ultraviolet transmissive composite fluorine resins, ultraviolet rays are prevented from being released to the outside. Furthermore, in the first and second ultraviolet transmissive composite fluorine resins of the stacking structure, the side to which the light-emitting material such as a pigment or a fluorescent member has adhered is coated or hermetically sealed with the ultraviolet transmissive and visible-light transmissive fluorine resin, and thus the light-emitting material is prevented from making contact with oxygen, with the result that it is possible to reduce or prevent degradation of the light-emitting material with oxygen. Therefore, as the emission of ultraviolet rays occurs stably within a fluorescent tube, it is possible to hold the light-emitting material to be excited in a stable state and effectively prevent or reduce degradation of the light-emitting material.

The fourth one can be said to be a composite general resin, in which a general resin material capable of blocking or absorbing ultraviolet rays is sandwiched between two fluorine resin materials. Examples of the general resin material described here include acrylic resin, polyethylene, polycarbonate, and a high-functionality resin having resistance to light and resistance to heat. Since these general resin materials have low durability against ultraviolet rays as is the case with a pigment or a fluorescent member, the general resin material is sandwiched between the two ultraviolet transmissive fluorine resins so as not to directly make contact with air. Thus, it is possible to prevent the general resin material from making contact with oxygen, and to reduce degradation of the resin material due to oxidation.

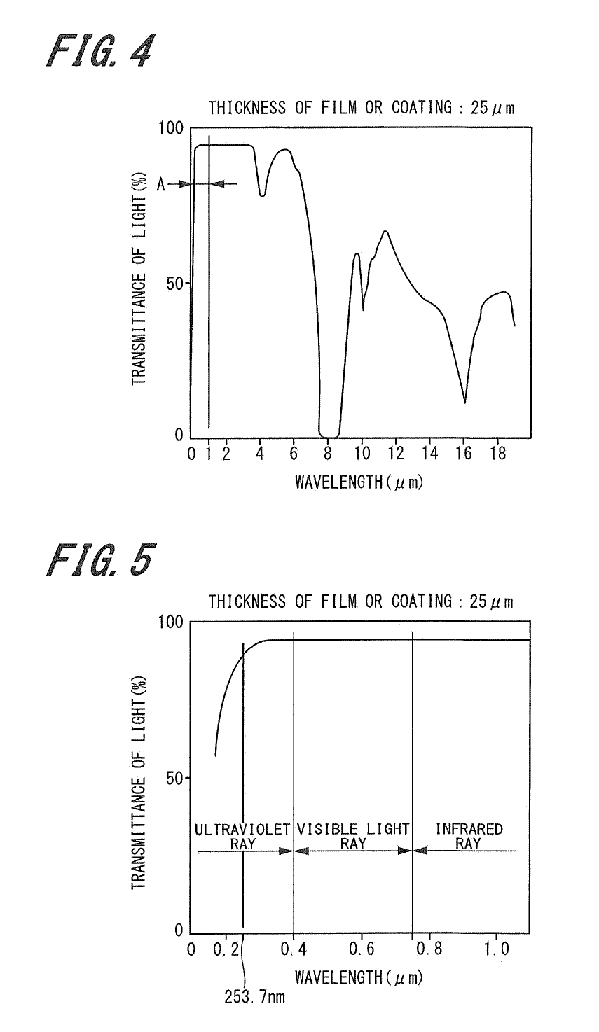

FIG. 4 shows exemplary light transmittance of films that have a thickness of 25 μm and that are formed of FEP (tetrafluoroethylene hexafluoropropylene copolymer) and PFA (polyfluoro alkoxy resin). The FEP and PFA resins are specific examples of the ultraviolet transmissive and visible-light transmissive fluorine resin, and are excellent in heat resistance, low temperature resistance, chemical resistance, electrical insulation, high frequent properties, and the like, and in particular have excellent qualities in light transmissiveness. FIG. 5 enlarges and shows a range A from a wavelength of 0 μm to a wavelength of 1.0 μm in FIG. 4. In the graph of FIG. 4, as the wavelength is increased, the light transmittance (%) rapidly rises from around a wavelength of 0 μm, and reaches the maximum of about 95% at a wavelength of about 0.3. In particular, as is clear from FIG. 5, the light transmittance rapidly rising from around a wavelength of 0 μm as the wavelength is increased reaches the approximate maximum point at a wavelength of 253.7 μm where the sterilization power of ultraviolet rays is the greatest.

When the ultraviolet-shielding resin member is obtained by including the light-emitting material such as a pigment or a fluorescent member or by sandwiching the general resin, since the light-emitting material and the general resin have low durability against ultraviolet rays in the air, it is necessary to use the light-emitting material and the general resin by setting an appropriate usage period, with consideration given to the degradation speed of the light-emitting material in the air and the like.

Thus, by coating one surface of the fluorine resin plate material or the fluorine resin film material with the light-emitting material such as a pigment or a fluorescent member, or by sandwiching the general resin with a plurality of ultraviolet transmissive and visible-light transmissive fluorine resins, and/or by further providing coating or sealing, even if the pigment, the fluorescent member, or the general resin is degraded by ultraviolet rays, it is possible to prevent occurrence of such trouble that the degraded and frazzled light-emitting material and the resin material leak to the outside and are scattered. In any one of the single fluorine resin, the mixture fluorine resin, and the composite fluorine resin, the thickness of the plate material or the film material is 3 mm (millimeters) or less; preferably, it is 1 mm or less with consideration given to the processbility.

In the ultraviolet-shielding plates 13a and 13b according to the first embodiment of the ultraviolet-shielding resin member of the present invention, shown in FIGS. 1 and 2, with consideration given to the poor processibility of the fluorine resin described in the “first one” discussed above, the ultraviolet non-transmissive and visible-light transmissive resin plate or film, or the ultraviolet transmissive and visible-light transmissive resin plate or film, which have been described in the second to fourth ones, are used. When the resin plate or the resin film described in the second to fourth ones are used, the thickness of the plate can be set at about 1 mm or 1 mm or less, and with this level of the plate thickness described above, it is possible to easily form a desired shape.

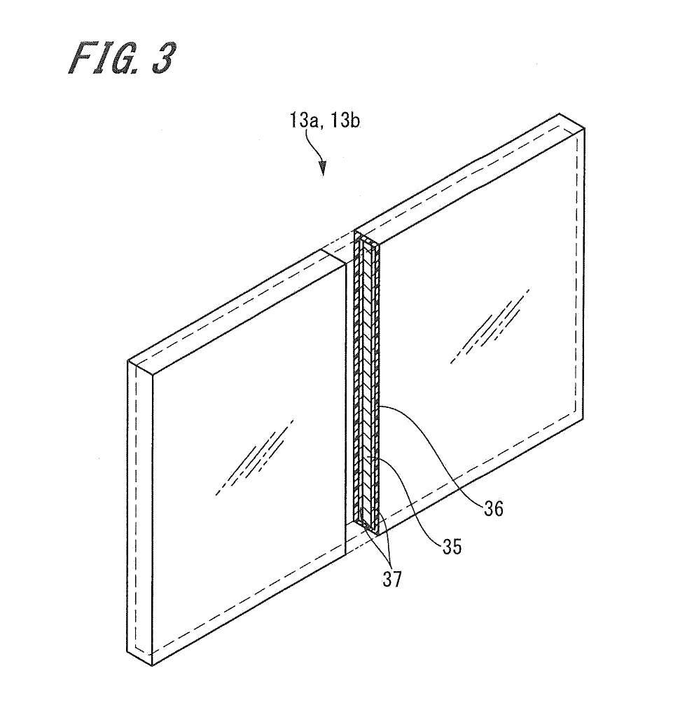

As shown in FIG. 3, the ultraviolet-shielding plates 13a and 13b include a plate substrate 35 that is formed of resin which transmits visible light rays but which does not transmit ultraviolet rays, and a film-shaped (or sheet-shaped) or coating external cover 36 that is formed of fluorine resin which transmits ultraviolet rays and that covers the entire surface of the plate substrate 35 without any gap. Furthermore, between both surfaces of the plate substrate 35 and the external cover 36, a light-emitting material layer 37 is provided by coating a fluorescent substance that converts the irradiated ultraviolet rays into visible light rays. When the light-emitting material layer is provided in the ultraviolet-shielding plates 13a and 13b as described above, it is possible to prevent degradation of the general resin, the pigment, and the like, and enhance the durability against ultraviolet rays. The light-emitting material layer 37 may be provided on only one side of the plate substrate 35. As the material of the plate substrate 35, for example, a light resistant synthetic resin that absorbs ultraviolet rays and does not transmit them, i.e., that is opaque to ultraviolet rays, can be used. When the light-emitting material is provided on both surfaces, as the plate substrate, a fluorine resin material or the like that is transparent to ultraviolet rays can be used. As the material of the external cover 36, for example, FEP or PFA resin can be applied.

The first ultraviolet-shielding plate 13a is formed such that its size is substantially the same as the lower surface portion 11a, and is configured such that its outer rim covers the outside of the frame portion of the lower surface portion 11a. Outside the first ultraviolet-shielding plate 13a, a lower surface holding plate 15a that has the same shape and size as the lower surface portion 11a is arranged, and, in the lower surface holding plate 15a, an opening hole 16 that has the same shape and size as the lower surface opening window 12a is provided.

In predetermined positions (in the present embodiment, in six places in total, that is, in four places in the four corners and in two places in the middle portion in the longitudinal direction) of the lower surface portion 11a, screw holes 17 for screwing the first ultraviolet-shielding plate 13a are provided. Corresponding to the screw holes 17 in the six places, six insertion holes 18 are provided in corresponding positions of the first ultraviolet-shielding plate 13a. Furthermore, in corresponding positions of the lower surface holding plate 15a, six insertion holes 19 are likewise provided. The screw shaft portions of fixing screws 21 are inserted into these insertion holes 18 and 19, and the screw shaft portions are screwed into the screw holes 17, and thus the first ultraviolet-shielding plate 13a is pressed by the lower surface holding plate 15a and is fixed to the lower surface portion 11a.

The second ultraviolet-shielding plate 13b is formed such that its size is substantially the same as the first side surface portion 11b, and is configured such that its outer rim covers the outside of the frame portion of the first side surface portion 11b. Outside the second ultraviolet-shielding plate 13b, a side surface holding plate 15b that has the same shape and size as the first side surface portion 11b is arranged, and in the side surface holding plate 15b, an opening hole 22 that has the same shape and size as the side surface opening window 12b is provided.

In predetermined positions (in the present embodiment, in six places in total, that is, in four places in the four corners and in two places in the middle portion in the longitudinal direction) of a pair of first side surface portions 11b, 11b, screw holes 23 for screwing the second ultraviolet-shielding plate 13b are provided. Corresponding to the screw holes 23 in the six places, six insertion holes 24 are provided in corresponding positions of the second ultraviolet-shielding plate 13b. Furthermore, in corresponding positions of the side surface holding plate 15b, six insertion holes 25 are likewise provided. The screw shaft portions of fixing screws 26 are inserted into these insertion holes 24 and 25, and the screw shaft portions are screwed into the screw holes 23, and thus the second ultraviolet-shielding plate 13b is pressed by the side surface holding plate 15b and is fixed to the first side surface portion 11b.

As shown in FIG. 1, on the inner surface of the pair of second side surface portions 11c, 11c, a pair of sockets 28 that removably mounts the ultraviolet lamp 5 is attached. Lead wires 29 drawn from the sockets 28 are connected to an unillustrated outlet, and electric power is fed from the outlet through the sockets 28 to the ultraviolet lamp 5. Hanging hardware 31 for hanging the casing 11 from a ceiling or the like is fixed to the four corners on the side of the opening portion 14 of the casing 11. The lower ends of a hanging unit are engaged with the hanging hardware 31.

The hanging unit 4 is formed of two hanging chains 4 each formed in a trifurcate shape having three support bar portions. In each of the hanging chains 4, two support bar portions are engaged with two pieces of hanging hardware 31, and the remaining one support bar portion is engaged with a hook fixed to the ceiling or the like. In this way, the lighting and air cleaning device 1 is used by being hung from a predetermined position of the ceiling or the like of a hospital, a house, a factory, etc.

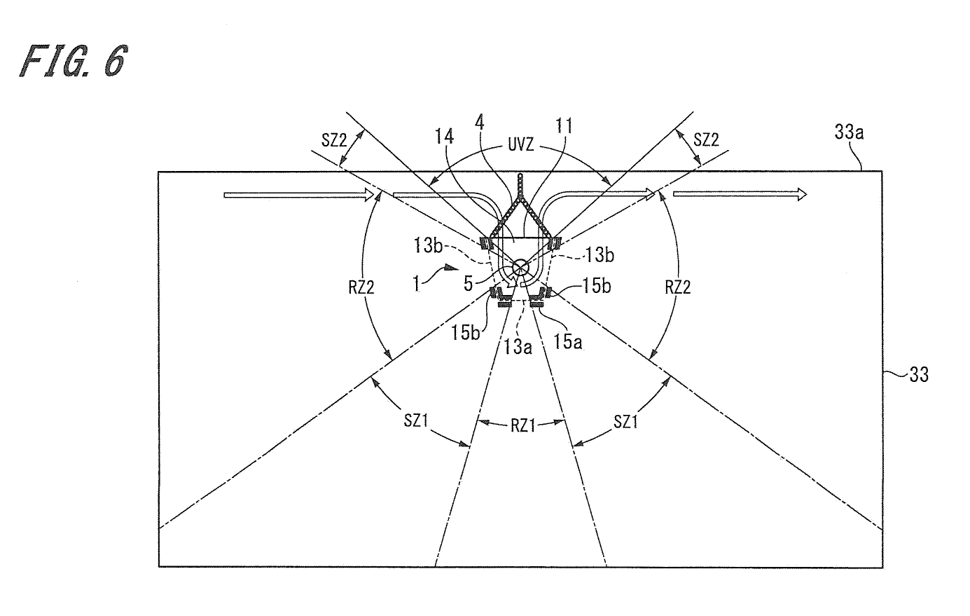

FIG. 6 is a diagram illustrating the effects of the lighting and air cleaning device 1 hung from a ceiling 33a of a building 33. When the ultraviolet lamp 5 is turned on in a condition that the lighting and air cleaning device 1 is hung, the electromagnetic wave emission portion of the lighting and air cleaning device 1 emits ultraviolet rays (about 15 to 30% of the total output) and visible light rays (about 50 to 60% of the total output) in various directions. The ultraviolet rays emitted from this electromagnetic wave emission portion kill or sterilize, within the casing 11, bacteria, viruses, and the like floating in the air present in the surrounding area of the electromagnetic wave emission portion, and thereby the air is cleaned.

Furthermore, heat (the remaining heat energy and the like other than the output of the ultraviolet rays and the visible light rays) generated when the ultraviolet lamp 5 emits ultraviolet rays and the like heats the surrounding air, and thus the natural convection of air is produced. Consequently, the air that has been heated to become light rises, and is discharged to the outside from the opening portion 14 provided in the upper portion of the casing 11, and, instead, air in the upper region that is heavyweight enters the casing 11. In this way, air is naturally convected in upward and downward directions, and bacteria, viruses, and the like floating in the newly-fed air are likewise killed or sterilized with the ultraviolet rays. By repeating the natural convection and the ultraviolet sterilization described above, it is possible to continuously clean the air within the building 33.

Among the ultraviolet rays emitted from the electromagnetic wave emission portion, the ultraviolet rays emitted upward through the opening portion 14 of the casing 11 to an ultraviolet emission region (a region to which the ultraviolet rays are directly irradiated) UVZ directly act on bacteria and viruses floating in the air within the ultraviolet emission region UVZ and kill or sterilize them. In this way also, it is possible to kill or sterilize bacteria, viruses, and the like floating in the air around the lighting and air cleaning device 1 and to clean the air within the building 33, and thus the sterilization effects by ultraviolet rays can be effectively exerted. Here, since the lighting and air cleaning device 1 is installed in a position higher than the head of human, the ultraviolet rays emitted upward from the electromagnetic wave emission portion are not irradiated directly to the human, and there is no fear that the human is affected by the ultraviolet rays.

At the same time, among the ultraviolet rays and the visible light rays emitted from the electromagnetic wave emission portion, the ultraviolet rays in a downward direction are blocked or absorbed by the first ultraviolet-shielding plate 13a attached to the lower surface portion 11a of the casing 11, whereas the visible light rays pass through the first ultraviolet-shielding plate 13a. Then, by the visible light rays emitted to a first visible light ray emission region (a region to which the visible light rays are irradiated) RZ1 that is formed by the lower surface opening window 12a of the lower surface portion 11a and the opening hole 16 of the lower surface holding plate 15a, a lower portion within the building 33 is mainly illuminated, and the surrounding area thereof can be illuminated.

Among the ultraviolet rays and the visible light rays emitted from the electromagnetic wave emission portion, the ultraviolet rays in a lateral direction are blocked or absorbed by the second ultraviolet-shielding plate 13b attached to the first side surface portions 11b of the casing 11, but the visible light rays pass through the second ultraviolet-shielding plate 13b. Then, by the visible light rays emitted to second visible light ray emission regions (regions to which the visible light rays are irradiated) RZ2 that are each formed of the side surface opening window 12b of the first side surface portions 11b and the opening hole 22 of the side surface holding plate 15b, side portions within the building 33 are mainly illuminated, and the surrounding areas thereof can be illuminated. Here, since the electromagnetic waves emitted from the first visible light ray emission region RZ1 and the second visible light ray emission regions RZ2 are not ultraviolet rays but visible light rays, a human is not affected by the visible light rays, and the illumination function of the lighting and air cleaning device 1 as an illumination unit can be effectively exerted.

On the other hand, due to the structure of the casing 11, a first shaded region (a region to which the visible light rays are not irradiated) SZ1 is formed between the first visible light ray emission region RZ1 and the second visible light ray emission region RZ2, and a second shaded region (a region to which the visible light rays are not irradiated) SZ2 is formed between the second visible light ray emission region RZ1 and the ultraviolet emission region UVZ. However, the first shaded region SZ1 is affected by the visible light rays emitted to the first visible light ray emission region RZ1 and the second visible light ray emission region RZ2 on both sides thereof, and thereby becomes bright, so that it is not affected by not being directly irradiated with visible light rays. Since the second shaded region SZ2 is not a region that needs to have brightness by the illumination device, it is also not affected by not being directly irradiated with visible light rays.

### Second Embodiment

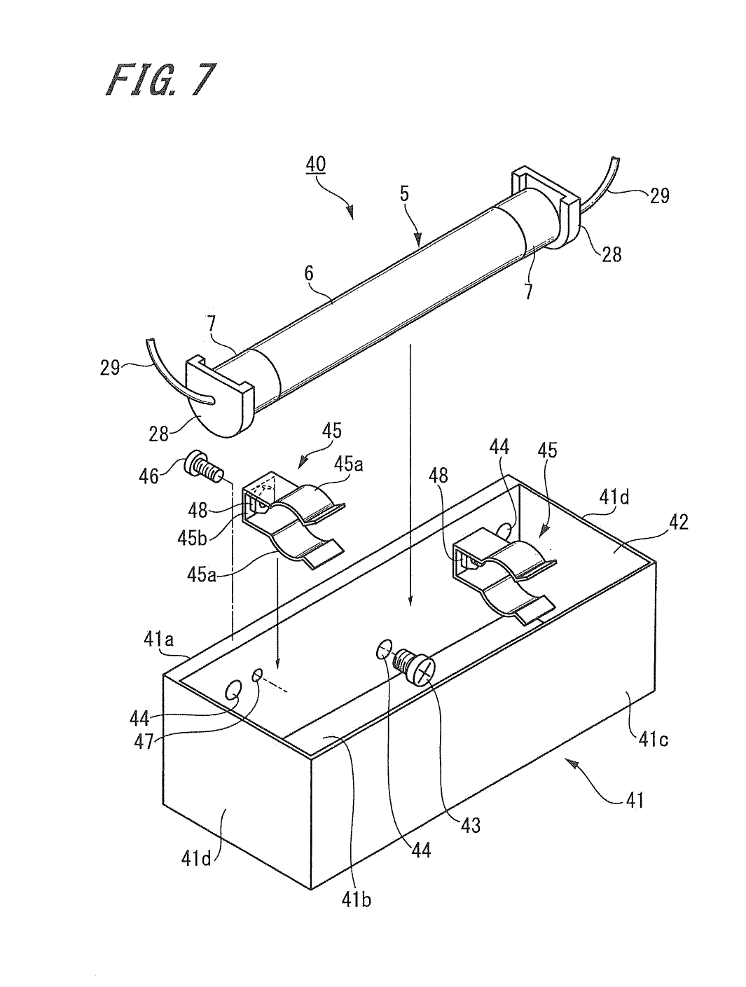

FIG. 7 is a diagram showing the second embodiment of the lighting and air cleaning device according to the present invention. A lighting and air cleaning device 40 shown in this embodiment includes the ultraviolet lamp 5 that is an electromagnetic wave generation source, and a cover and casing 41 that indicates a second specific example of the ultraviolet-shielding resin member. The cover and casing 41 is formed of a horizontally long enclosure having an opening portion 42 in an upper surface. The cover and casing 41 has a horizontally long rear surface portion 41a that serves as a fixation base, a horizontally long lower surface portion 41b that continuously protrudes forward on the lower portion of the rear surface portion 41a, a front surface portion 41c that continuously rises upward on the front portion of the lower surface portion 41b, and a pair of side surface portions 41d, 41d that rises continuously on both sides in the longitudinal direction.

The entire cover and casing 41 is integrally formed of an ultraviolet non-transmissive and visible-light transmissive resin material that blocks or absorbs ultraviolet rays and that transmits visible light rays. In this embodiment, an ultraviolet-shielding plate obtained by further coating the surface of the mixture fluorine resin, the composite fluorine resin or the composite general resin including the light-emitting material (a pigment or a fluorescent member) with the ultraviolet transmissive and visible-light transmissive fluorine resin is used, however, the single ultraviolet transparent fluorine resin can also be used. In this case, when the ultraviolet-shielding resin member of the single ultraviolet transparent fluorine resin is used, the thickness of the cover and casing is set at about 5 mm in all parts.

In the cover and casing 41 described in this embodiment, its strength is set relatively great by making only the thickness of the rear surface portion 41a greater than those of the other portions. The rear surface portion 41a is fixed to a wall or the like with fixing screws 43, and thus the lighting and air cleaning device 40 can be fixed to a predetermined position of a wall surface of a building or the like. Hence, in the rear surface portion 41a, a plurality of (in this embodiment, three) insertion holes 44, through which the screw shaft portions of the fixing screws 43 are inserted, and a plurality of (in this embodiment, two) insertion holes 47 through which the screw shaft portions of fixing screws 46 are inserted to attach lamp holding units 45, are provided.

The lamp holding units 45 are each a unit for holding the ultraviolet lamp 5, and are fixed, at two places, to the rear surface portion 41a, with the fixing screws 46. The lamp holding unit 45 is formed of a ribbon-shaped plate spring material having appropriate elasticity, and includes a pair of holding parts 45a, 45a that holds the ultraviolet lamp 5, and a fixing part 45b that couples the holding parts 45a, 45a together. The pair of holding parts 45a, 45a is formed by bending both sides of the fixing part 45b at 90° and making the sides to face each other. A nut 48 is fixed to the inside of the fixing part 45b, and the screw shaft portion of the fixing screw 46 penetrating the insertion hole 44 of the rear surface portion 41a is screwed through the nut 48. The lamp holding unit 45 is fixed to the inner surface of the rear surface portion 41a by tightening the fixing screw 46. Counter borings for accommodating head portions of the fixing screws 46 are provided in rear surface portion 41a outside of the insertion holes 44.

The ultraviolet lamp 5 is held to the cover and casing 41 with the two lamp holding units 45, 45 fixed to the rear surface portion 41a. The sockets 28 are mounted to the ferrules 7, 7 at both ends of the ultraviolet lamp 5, and electric power is fed through lead wires 29.

In the lighting and air cleaning device 40 according to the second embodiment, the effects of the opening portion 42 of the cover and casing 41 are the same as in the ultraviolet-shielding resin member 3 of the first embodiment, and it is possible to produce natural convection of air around the ultraviolet lamp 5 and to apply ultraviolet rays directly to air in a higher place. It is therefore possible to kill or sterilize bacteria, viruses, and the like floating in the surrounding air and to thereby clean the air around the lighting and air cleaning device 40.

On the other hand, since the whole portions of the rear surface portion 41a, the lower surface portion 41b, the front surface portion 41c, and the pair of side surface portions 41d, 41d, other than the opening portion 42, of the cover and casing 41 are formed of ultraviolet non-transmissive and visible-light transmissive resin, electromagnetic waves are emitted from all the surfaces other than the rear surface portion 41a fixed to a wall or the like. Since the electromagnetic waves emitted from each of the surface portions are not ultraviolet rays but visible light rays, a human is not affected by the visible light rays, and the illumination function of the lighting and air cleaning device 40 as an illumination unit can be exerted.

### Third Embodiment

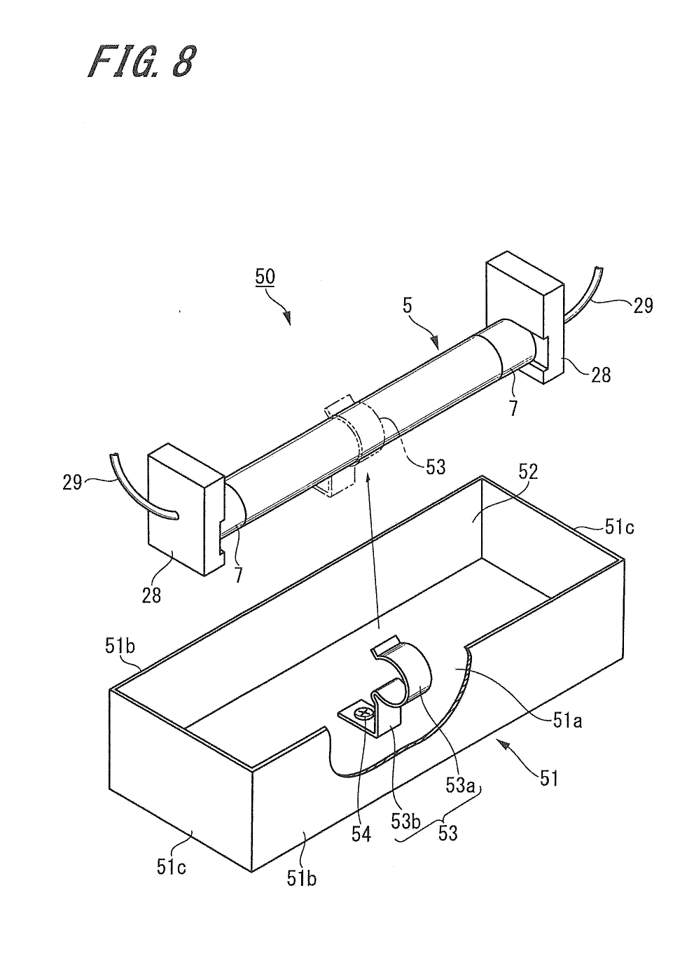

FIG. 8 is a diagram showing the third embodiment of the lighting and air cleaning device according to the present invention. A lighting and air cleaning device 50 described in this embodiment includes the ultraviolet lamp 5 that is an electromagnetic wave generation source, and a cover enclosure 51 showing a third specific example of the ultraviolet-shielding resin member. The lighting and air cleaning device 50 according to the third embodiment differs from the lighting and air cleaning device 40 according to the second embodiment in that a pair of sockets 28, 28 is fixed to predetermined positions of a building or the like, and that the cover enclosure 51 can be hung from the ultraviolet lamp 5 held between the pair of sockets 28, 28. Hence, the cover enclosure 51 will be described here, and overlapped description will not be repeated, with the same parts as in the first and second embodiments identified with like symbols.

The cover enclosure 51 is formed of a horizontally long cover member having an opening portion 52 in an upper surface. The cover enclosure 51 has a horizontally long lower surface portion 51a, a pair of horizontally long first side surface portions 51b, 51b that rises continuously on both sides of the lower surface portion 51a in the width direction, and a pair of second side surface portions 51c, 51c that rises continuously on both sides of the lower surface portion 51a in the longitudinal direction. As with the cover and casing 41, the entire cover enclosure 51 is integrally formed of ultraviolet-shielding resin that blocks or absorbs ultraviolet rays and that transmits visible light rays. Substantially in the center portion of the inner surface of the lower surface portion 51a, an attachment hook 53 for hanging and supporting the cover enclosure 51 from and to the ultraviolet lamp 5 is screwed and fixed with a fixing screw 54.

The attachment hook 53 has a hook portion 53a that is hooked around the glass tube 6 of the ultraviolet lamp 5, and a fixation portion 53b that is continuously and integrally formed at one end of the hook portion 53a. The attachment hook 53 is formed of a ribbon-shaped plate spring material having an appropriate width. The hook portion 53a of the attachment hook 53 is formed of an arc-shaped portion that is curved to have substantially the same radius of curvature as the external diameter of the glass tube 6. One end of the L-shaped fixation portion 53b is continuous to one end of the hook portion 53a. An insertion hole is provided in one part of the fixation portion 53b. A screw shaft portion inserted through the insertion hole is screwed through a screw hole provided in the lower surface portion 51a and is tightened, and thus the attachment hook 53 is screwed and fixed to substantially the center portion of the lower surface portion 51a.

In the lighting and air cleaning device 50 according to the third embodiment, the pair of sockets 28, 28 is fixed to predetermined positions of a ceiling or the like. The attachment hook 53 is hooked around the glass tube 6 of the ultraviolet lamp 5 placed between the pair of sockets 28, 28, and thus the cover enclosure 51 is attached to the ultraviolet lamp 5 that is a light source.

The effects of the cover enclosure 51 are substantially the same as the cover and casing 41 according to the second embodiment but differ from it in that all the surfaces of the cover enclosure 51 other than the opening portion 52 constitute the visible light ray emission portion. Hence, in this embodiment, the visible light ray emission portion is increased, and thus it is possible to accordingly increase the efficiency of irradiation of visible light rays when the lighting and air cleaning device 50 serves as an illumination unit. In this embodiment, since the total weight of the cover enclosure 51 is applied to the ultraviolet lamp 5, it is preferable that the thickness of the cover enclosure 51 is minimized and thus the weight is reduced. Hence, in this embodiment, it is undesirable to use, as the material of the cover enclosure 51, the ultraviolet non-transmissive and visible-light transmissive resin that can constitute an ultraviolet-shielding resin member by making the thickness thereof to 5 mm or more, because the weight is increased accordingly.

### Fourth Embodiment

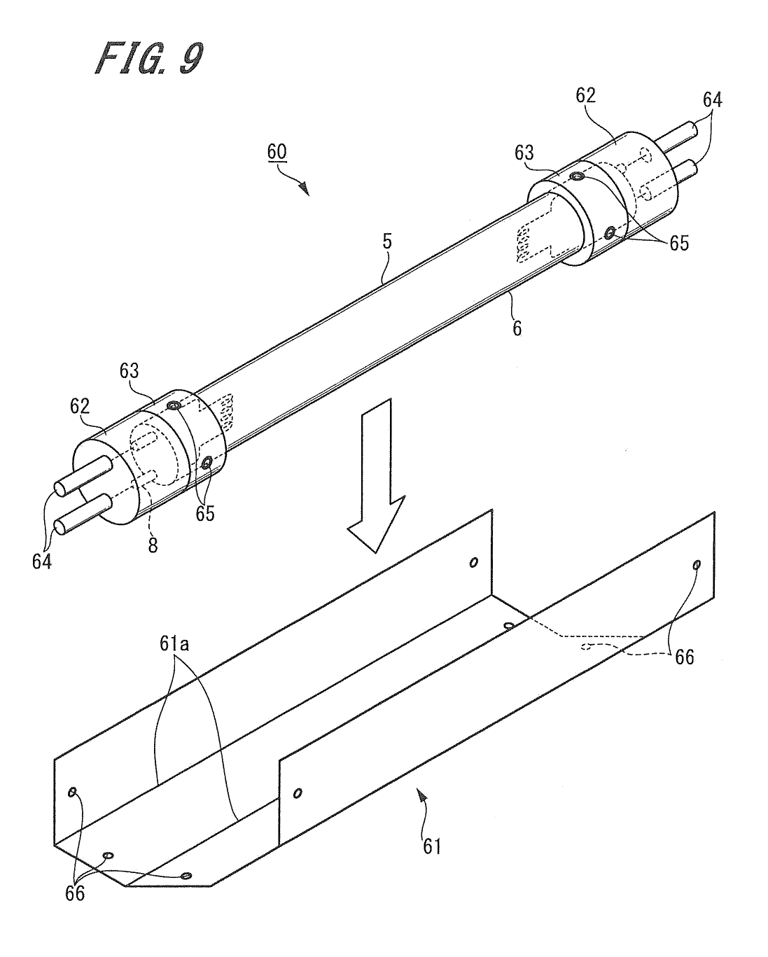

FIGS. 9 to 15 are diagrams showing the fourth embodiment and its variations of the lighting and air cleaning device according to the present invention. A lighting and air cleaning device 60 according to the fourth embodiment, shown in FIGS. 9 to 11, includes the ultraviolet lamp 5 that is an electromagnetic wave generation source, a cover tube 61 showing a fourth specific example of the ultraviolet-shielding resin member, a pair of ferrule adaptors 62, 62 for fitting the ultraviolet lamp 5 into different-size sockets, and attachment members 63 for attaching the cover tube 61 to the ultraviolet lamp 5.

As shown in FIG. 9, the ultraviolet lamp 5 is the one manufactured based on the standard specifications for an ultraviolet lamp, and the diameter and length of the glass tube 6, the diameter and length of the ferrules 7, the diameter and the length of the ferrule pins 8, and the like are all formed according to specified dimensions. As compared with the ultraviolet lamp 5, a fluorescent tube used as a light source for home illumination is manufactured based on its specific standard specifications different from the standard specifications for the ultraviolet lamp. Hence, various dimensions such as the diameter and length of the fluorescent tube are different from those of the ultraviolet lamp 5.

Therefore, in a general household illumination unit using a fluorescent tube, it is impossible to use the ultraviolet lamp 5 as it is, instead of the fluorescent tube. Hence, it is the ferrule adaptors 62 that are used in an ordinary home to enable using a standard-size ultraviolet lamp as is. By mounting the ferrule adaptors 62 to the ferrules 7, 7 at both ends of the ultraviolet lamp 5, it becomes possible to use, as with a general fluorescent tube, the lighting and air cleaning device 60 that is formed of the ultraviolet lamp 5 incorporating the ferrule adaptors 62.

The ferrule adaptor 62 has a tubular ferrule body whose one end is closed, and two connection pins 64, 64 are provided on the closed end surface of the ferrule body. The two connection pins 64, 64 have a shape and dimensions corresponding to connection terminals of a socket in a fluorescent tube illumination device, and can be connected and separated to and from the connection terminals. On the side opposite to the connection pins 64 of the ferrule body, a recess portion into which the ferrule 7 of the ultraviolet lamp 5 is fitted is provided. The ferrule 7 is fitted into the recess portion, and thus the two ferrule pins 8, 8 protruding from one end of the ferrule 7 are electrically connected to the two connection pins 64, 64.

The attachment members 63 are each formed as an attachment ring that is ring-shaped to fit into the glass tube 6. The attachment rings 63 fit the glass tube 6, and thus are attached to the ultraviolet lamp 5. In the attachment rings 63, a plurality of screw holes (in this embodiment, four) 65 for screwing the cover tube 61 are spaced regularly in a circumferential direction. The attachment rings 63 may be removably attached to the glass tube 6 or may be adhered thereto with an adhesive or the like.

The cover tube 61 is formed of a film material made of ultraviolet non-transmissive and visible-light transmissive resin that blocks or absorbs ultraviolet rays and that transmits visible light rays. A light-emitting material layer is preferably provided on the entire surface of the cover tube 61 opposite the ultraviolet lamp 5. As described above, by providing the light-emitting material layer on the cover tube 61, it becomes possible to prevent or inhibit degradation of the cover tube 61 itself, and to increase the illumination efficiency in the visible light ray emission portion by converting ultraviolet rays into visible light rays, thereby illuminating the surrounding area more brightly.

In this embodiment, the cover tube 61 is configured as a quadrangular tube by bending one ultraviolet-shielding resin plate (or film) at a plurality of places (in this embodiment, four places). Hence, in each side in a direction in which folding lines 61a of the cover tube 61 extend, five insertion holes 66 are provided. The cover tube 61 may be formed as a quadrangular tube having no cut portions in a circumferential direction. Needless to say, the cover tube 61 may be formed as a triangular tube, a pentangular tube, a hexagonal tube, an octagonal tube or another tube.

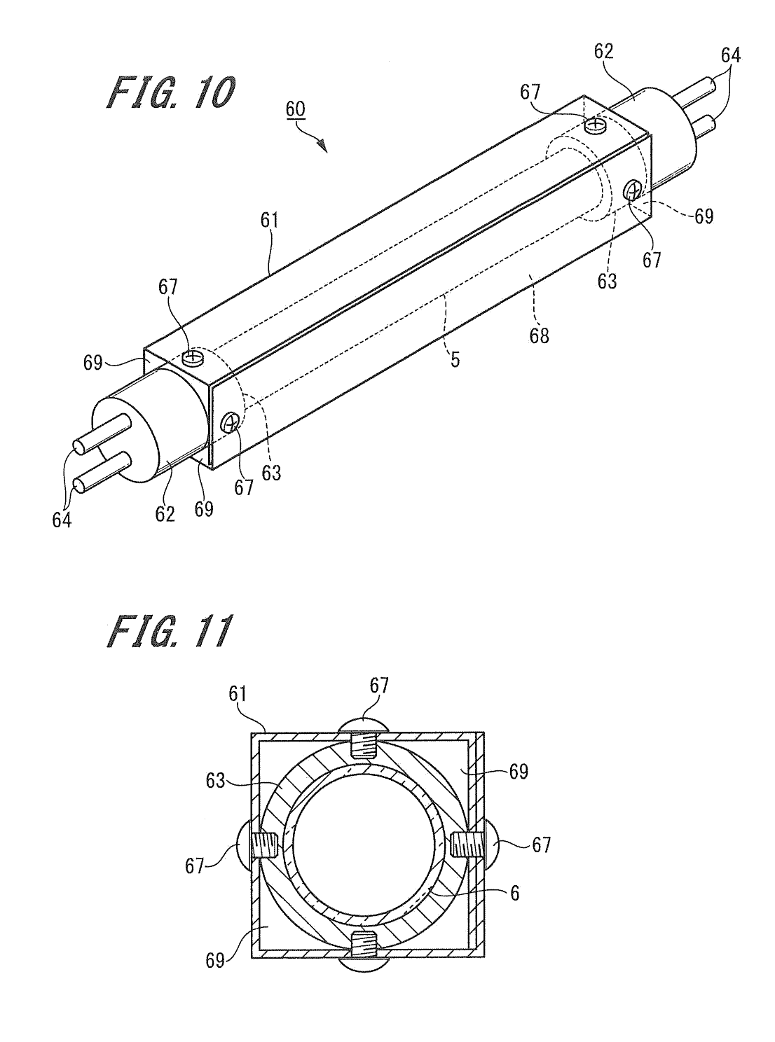

FIG. 10 is a diagram showing the overall configuration of the lighting and air cleaning device 60; the cover tube 61 which is formed in the shape of a quadrangular tube by being bent and whose cross section is quadrangular is attached to the attachment rings 63 with fixing screws 67. The length of the cover tube 61 in the axial direction is set substantially equal to the length from one of the attachment rings 63 fitted into one end of the ultraviolet lamp 5 in the axial direction to the other attachment ring 63 fitted into the other end. A space portion provided between the cover tube 61 and the ultraviolet lamp 5 forms an air passage 68.

The air passage 68 is a space through which the air around the ultraviolet emission portion moves, and space portions provided between the inner surface of the cover tube 61 and the outer circumferential surfaces of the pair of attachment rings 63, 63 form two exit and entrance ports 69. Specifically, as shown in FIG. 11, the length of one side of the cover tube 61 is set substantially equal to the outer diameter of the attachment ring 63, and the intermediate portions of the four sides are fixed to the attachment rings 63 with the fixing screws 67, and thus four exit and entrance ports 69 whose one side is arc-shaped and whose cross section is substantially triangular are provided in both ends of the ultraviolet lamp 5 in the axial direction.

The effects of the lighting and air cleaning device 60 configured as described above are as follows. Since the lighting and air cleaning device 60 has the pair of ferrule adaptors 62, 62 at both ends of the ultraviolet lamp 5 in the axial direction, in a hospital, a food factory, and the like where a sterilization device using a ultraviolet lamp is necessary, the lighting and air cleaning device 60 can be used as with a general ultraviolet tube used as an illumination device for an ordinary home. In other words, for example, the lighting and air cleaning device 60 is used by being mounted to an illumination device arranged in a predetermined position of a ceiling, a wall or the like.

When electric power is fed to the lighting and air cleaning device 60, and thus the ultraviolet lamp 5 is turned on, ultraviolet rays and visible light rays are emitted from the electromagnetic wave emission portion in various directions. By the ultraviolet rays emitted from the electromagnetic wave emission portion, bacteria, viruses, and the like floating in the air within the air passage 68 formed in the cover tube 61 are killed or sterilized, and thus the air is cleaned. Here, since the surrounding air is warmed by heat generated by the ultraviolet lamp 5 and becomes light, heavy air outside the cover tube 61 enters the air passage 68 through the exit and entrance ports 69, and, instead, the light air is pushed out of the air passage 68 through the exit and entrance ports 69 to the outside. In this way, natural convection occurs in which internal and external air flows through the air passage 68 within the lighting and air cleaning device 60, and thus sterilization by ultraviolet rays on the air passing through the air passage 68 is continuously performed.

At the same time, the visible light rays emitted from the electromagnetic wave emission portion pass through the cover tube 61 covering the entire circumference of the electromagnetic wave emission portion, and are emitted from the entire surface to the outside. Here, when the cover tube 61 is formed of the previously described single ultraviolet transmissive fluorine resin or the composite ultraviolet transmissive fluorine resin, the entire cover tube 61 emits light according to the color of a slightly whitish pigment contained in the ultraviolet transmissive and visible-light transmissive fluorine resin or the color of a resin sandwiched between the fluorine resins, and the light of such a color illuminates the surrounding area. In this way, since the entire cover tube 61 functions as the visible light ray emission portion that emits visible light rays, and illuminates the surrounding area, the illumination effects of the lighting and air cleaning device 60 are exerted.

In the fourth embodiment, since the ultraviolet-shielding plate is bent along the predetermined folding line 61a, and thus the cover tube 61 is configured, it is possible to simply and rapidly perform an assembly operation, and to reduce the occupied volume before the assembly and thereby increase the efficiency of transport. Moreover, the assembly operation of the cover tube 61 is only screwing both end portions in the axial direction with the fixing screws 67, and by performing only the screwing operation described above, it is possible to form the air passage 68 having the exit and entrance ports 69 at both ends. It is therefore possible to simplify the configuration of this type of lighting and air cleaning device and ease the assembly operation.

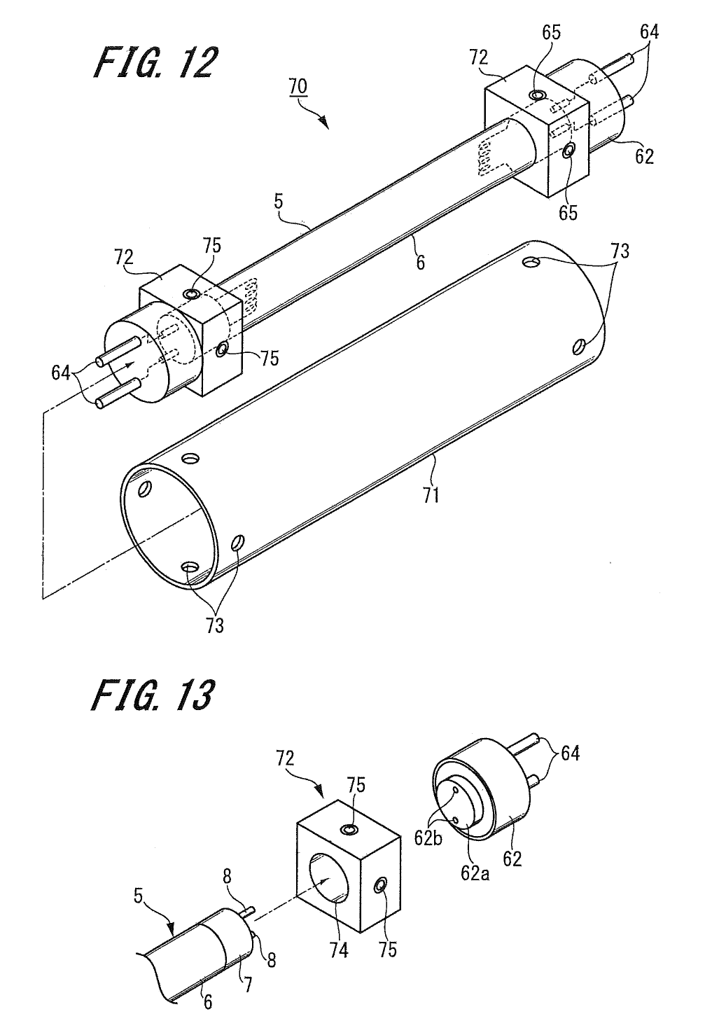

FIGS. 12 to 14 are diagrams showing a first variation of the fourth embodiment of the lighting and air cleaning device according to the present invention. A lighting and air cleaning device 70 showing the first variation of the fourth embodiment differs from the lighting and air cleaning device 60 described above in that the ultraviolet-shielding resin member is formed of a cover cylinder 71 whose cross section is circular, and that attachment members are formed of attachment blocks 72 whose cross section is quadrangular. Hence, the cover cylinder 71 and the attachment blocks 72 will be described here, and overlapped description will not be repeated, with other like parts in the configuration identified with like symbols.

As shown in FIG. 12, the cover cylinder 71 is formed of the ultraviolet non-transmissive and visible-light transmissive fluorine resin that blocks or absorbs ultraviolet rays and that transmits visible light rays as a cylinder whose cross section is circular. This cover cylinder 71 may be formed as a cylinder that is highly rigid and that is unlikely to be deformed, or by contrast, the cover cylinder 71 may be formed as a cylinder that is highly flexible and that is easily subjected to elastic deformation. On substantially entire inner surface of the cover cylinder 71 opposite the ultraviolet lamp 5, a light-emitting material layer obtained by applying, in a layer, a fluorescent member which is excited by irradiation of ultraviolet rays to emit visible light rays is preferably provided. Since the light-emitting material layer is coated with the ultraviolet transmissive fluorine resin material, and thus the light-emitting material layer is prevented from making contact with air, it is possible to stably emit light and enhance the durability. In this case, the coating can be realized by performing the operation under an environment of a deaeration or vacuum state or under an environment where an inert gas is purged. Four insertion holes 73 are provided in each end of the cover cylinder 71 in the axial direction.

As shown in FIG. 13, the attachment block 72 is formed of a quadrangular block member having a round penetration hole 74 into which the ferrule 7 of the ultraviolet lamp 5 is fitted. From one side, the ferrule 7 of the ultraviolet lamp 5 is fitted into the penetration hole 74 of the attachment block 72, and from the other side, a cylindrical fitting projection portion 62a provided in one side surface of the ferrule adaptor 62 is fitted thereinto. In the end surface of the fitting projection portion 62a, two fitting holes 62b, 62b into which the two ferrule pins 8 provided on the ferrules 7 are fitted are provided. The ferrule 7 and the fitting projection portion 62a are fitted from both sides into the attachment block 72, and thus the two ferrule pins 8 are fitted into the two fitting holes 62b, 62b within the attachment block 72. In this way, the two ferrule pins 8 and the two connection pins 64, 64 are electrically connected to each other.

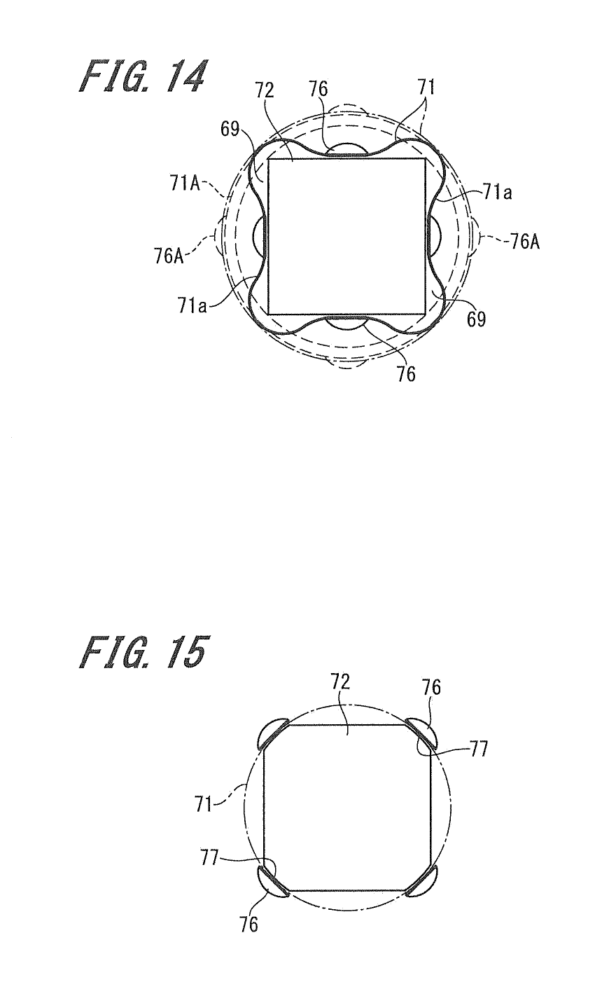

In the four surfaces of the attachment block 72, the screw holes 75 into which the cover cylinder 71 is screwed with the fixing screws are provided. The form of attachment of the cover cylinder 71 differs depending on whether the cover cylinder 71 is formed of a highly flexible resin material or the cover cylinder 71 is formed of a highly rigid resin material. FIG. 14 is a diagram illustrating cases where the rigidity of the cover cylinder 71 differs.

In FIG. 14, a case where the cover cylinder 71 is formed of a highly flexible resin material is indicated by a solid line and an alternate long and short dashed line. In this case, using fixing screws 76 having short pin portions, four fixation portions at four places are tightened, thus the end portion of the cover cylinder 71 is curved at four places, and an arc-shaped projection portion 71a is formed between the adjacent fixing screws 76. An inside space portion of the arc-shaped projection portion 71a forms the exit and entrance port 69 of the air passage 68. On the other hand, a cover cylinder 71A indicated by a broken line indicates a case where it is formed of a highly rigid resin material. Here, using fixing screws 76A having long pin portions, four fixation portions at four places are tightened, and thus the rear surfaces of the head portions of the fixing screws 76A are pressed onto the outer circumferential surface of the cover cylinder 71A. In this case, a semicircular space portion formed between the inner surface of the cover cylinder 71A and the outer surface of the attachment block 72 forms the exit and entrance port 69 of the air passage 68.

FIG. 15 is a diagram showing another form of attachment of the cover cylinder 71 to the attachment blocks 72. In this form, in the four corners of the attachment block 72, arc-shaped screwing surfaces 77 are provided, and the inner diameter of the cover cylinder 71 is set such that the arc-shaped screwing surfaces 77 fit the attachment block 72. In each of the arc-shaped screwing surfaces 77 of the attachment block 72, a screw hole into the fixing screw 76 is screwed is provided. With this configuration, it is possible to obtain the same effects as in the forms of attachment described above.

### Fifth Embodiment

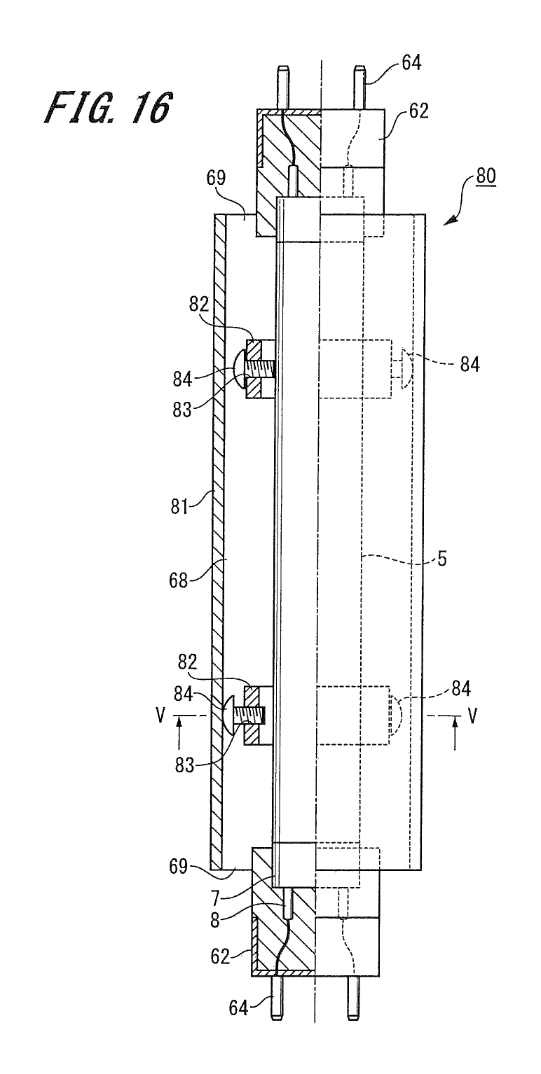

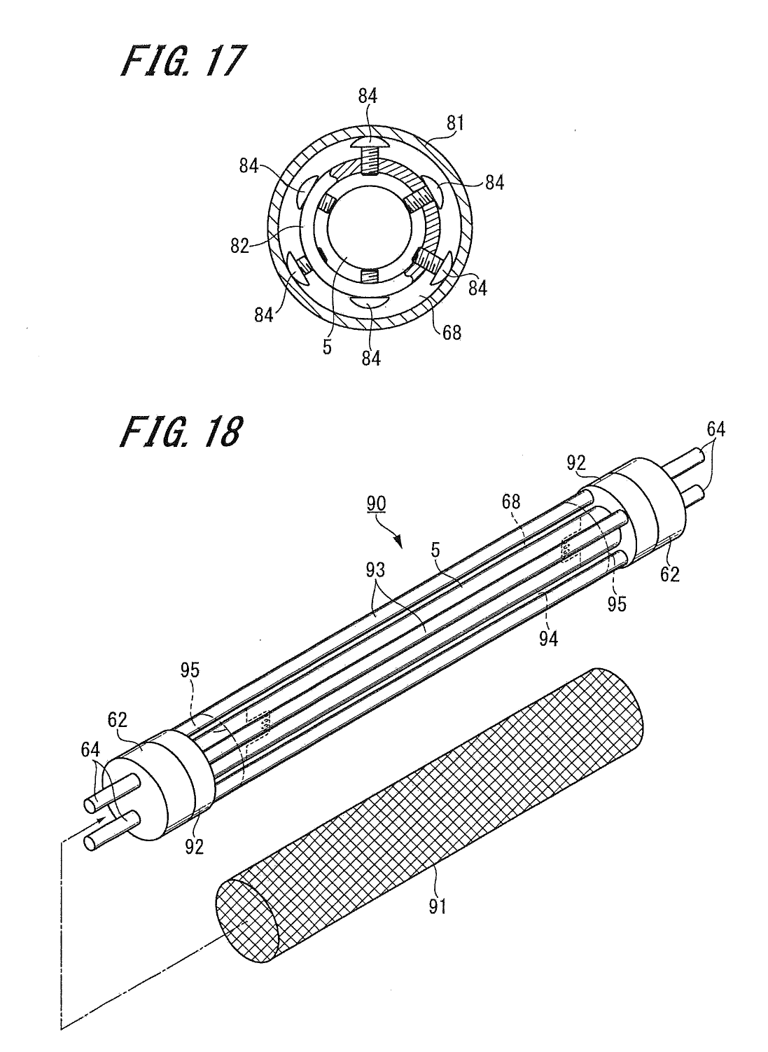

FIGS. 16 and 17 are diagrams showing a fifth embodiment of the lighting and air cleaning device according to the present invention. A lighting and air cleaning device 80 according to the fifth embodiment includes the ultraviolet lamp 5 that is an electromagnetic wave generation source, a cover tube 81 that indicates a fifth specific example of the ultraviolet-shielding resin member the pair of ferrule adaptors 62, 62 for fitting the ultraviolet lamp 5 into a different-sized socket, and two support rings 82, 82 for supporting the cover tube 81 to the ultraviolet lamp 5.

The lighting and air cleaning device 80 according to the fifth embodiment differs from the lighting and air cleaning device 60 described above in that the ultraviolet-shielding resin member formed of the cover tube 81 whose cross section is circular is supported with the two support rings 82, 82. Hence, here, the support rings 82 will be described in detail, and overlapped description will be given in short or will not be repeated, with like parts identified with like symbols.

As shown in FIGS. 16 and 17, the support ring 82 is formed of a ring-shaped member held within the air passage 68, and is formed such that its inner diameter is larger than the outer diameter of the glass tube 6 of the ultraviolet lamp 5, and its outer diameter is smaller than the inner diameter of the cover tube 81. In the support ring 82, six screw holes 83 are provided at equal intervals in a circumferential direction. An adjustment screw 84 is screwed into each of the screw holes 83 with its head portion on the outside. By adjusting the amount of protrusion of the head portion of the adjustment screw 84, the space with the glass tube 6 and the space with the cover tube 81 are individually adjusted, and thereby the cover tube 81 can be supported without becoming loose. With this configuration, it is also possible to obtain the same effects as in the embodiments described above.

### Sixth Embodiment

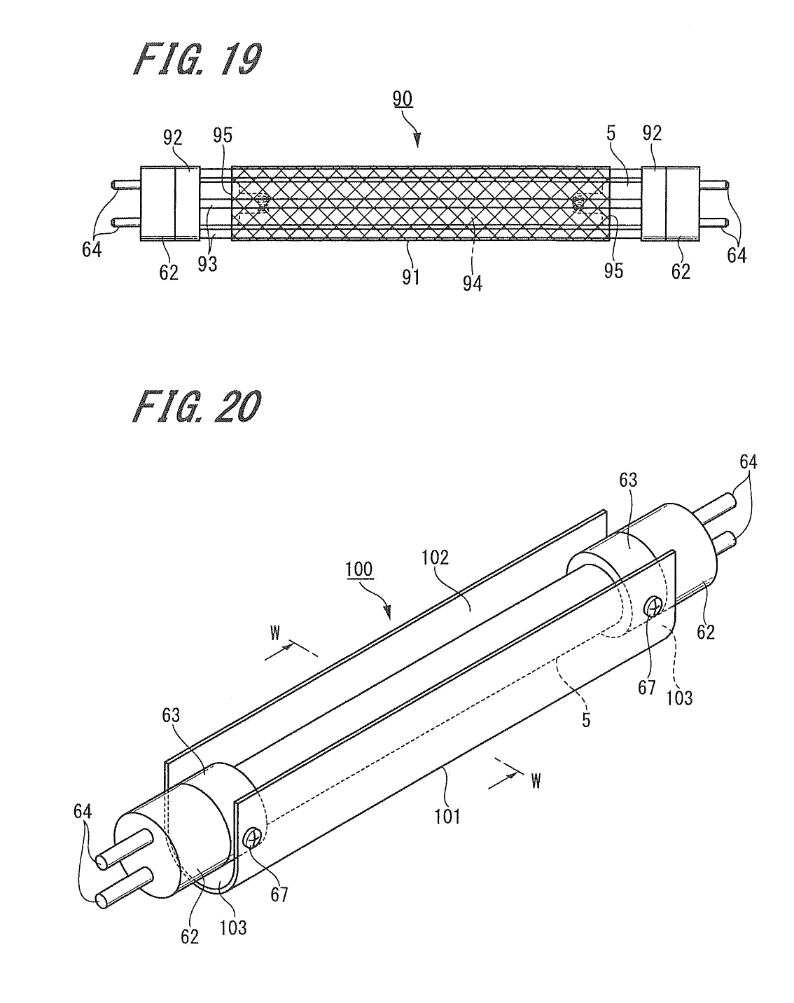

FIGS. 18 and 19 are diagrams showing a sixth embodiment of the lighting and air cleaning device according to the present invention. A lighting and air cleaning device 90 according to the sixth embodiment includes the ultraviolet lamp 5 that is an electromagnetic wave generation source, a cover tube 91 that is an ultraviolet-shielding resin member, the pair of ferrule adaptors 62, 62 for fitting the ultraviolet lamp 5 into a different-sized socket, two attachment rings 92, 92 for supporting the cover tube 91 to the ultraviolet lamp 5, and a plurality of cover support bars 93 that are placed between the two attachment rings 92, 92.

The lighting and air cleaning device 90 according to the sixth embodiment differs from the lighting and air cleaning device 60 described above in that the ultraviolet-shielding resin member formed of the cover tube 91 whose cross section is circular is supported with the plurality of cover support bars 93 placed between the two attachment rings 92, 92. Hence, here, the attachment rings 92 and the cover support bars 93 will be described in detail, and overlapped description will be given in short or will not be repeated, with like parts identified with like symbols.

As shown in FIGS. 18 and 19, the pair of attachment rings 92 is arranged on the inside of the ferrule adaptors 62, 62, and the four cover support bars 93 are placed therebetween. The four cover support bars 93 are regularly spaced so as to cover the ultraviolet lamp 5, and the cover tube 91 is supported by these four cover support bars 93. The cover tube 91 is formed such that the length in the axial direction is less than that of the cover support bar 93 in the axial direction. In this way, a gap is formed between both ends of the cover tube 91 in the axial direction and the pair of attachment rings 92, 92, and this gap is used as exit and entrance ports 95 of an air passage 94.

In the lighting and air cleaning device 90 according to the sixth embodiment configured as described above, as in the embodiment described above, it is also possible to kill bacteria, viruses, and the like floating in the air passing through the interior of the air passage 94, and clean the air. In this embodiment, since the length of the cover tube 91 in the axial direction is less than that of the glass tube 6 of the ultraviolet lamp 5, ultraviolet rays may leak through the exit and entrance ports 95 at both ends of the air passage 94. However, a structure is adopted in which the end portions of the cover tube 91 extend to stem glass holding the electrodes of the ultraviolet lamp 5 to cover the electrodes. In this way, it is possible to displace the exit and entrance ports 95 of the air passage 94 from the position of the electrodes, and minimize leakage of ultraviolet rays through the exit and entrance ports 95.

### Seventh Embodiment

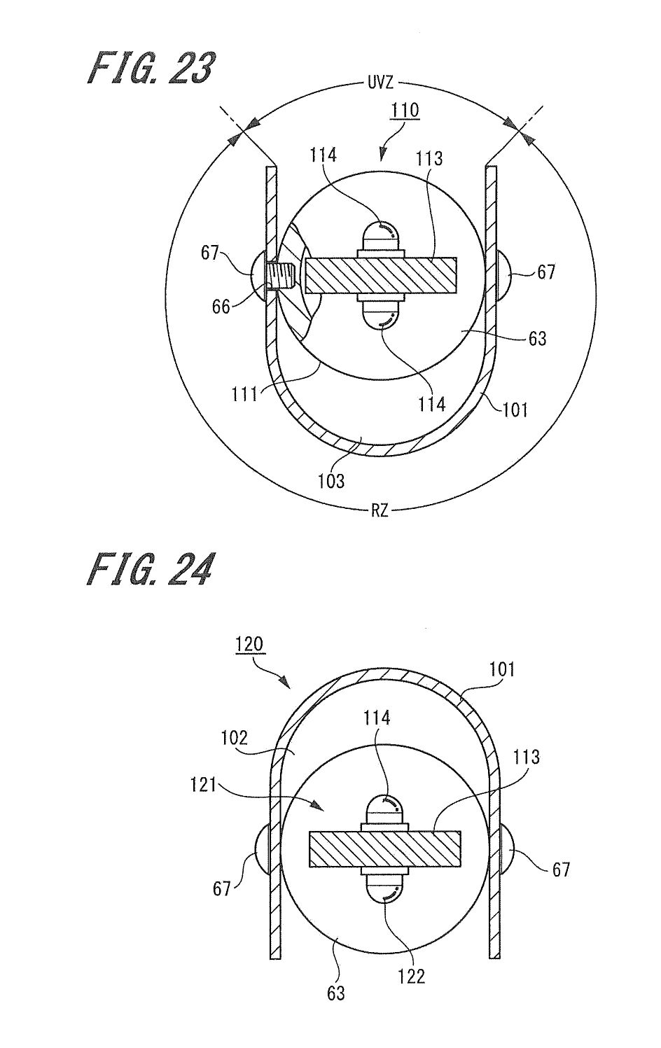

FIGS. 20 to 27 are diagrams showing a seventh embodiment and its variation of the lighting and air cleaning device according to the present invention. A lighting and air cleaning device 100 according to the seventh embodiment, shown in FIGS. 20 and 21, includes the ultraviolet lamp 5 that is an electromagnetic wave generation source, a gutter-like cover member 101 that indicates a fifth specific example of the ultraviolet-shielding resin member, the pair of ferrule adaptors 62, 62 for fitting the ultraviolet lamp 5 into a different-sized socket, and a pair of attachment rings 63, 63 for attaching the gutter-like cover member 101 to the ultraviolet lamp 5.

The lighting and air cleaning device 100 according to the seventh embodiment differs from the lighting and air cleaning device 60 described above in that the gutter-like cover member 101 whose cross section is U-shaped is configured as the fifth specific example of the ultraviolet-shielding resin member. Hence, here, the gutter-like cover member 101 will be described in detail, like parts are identified with like symbols, and overlapped description will be given in short or will not be repeated. The ultraviolet lamp 5, the pair of ferrule adaptors 62, 62, and the pair of attachment rings 63, 63 are the same as those described before.

As shown in FIG. 20, the gutter-like cover member 101 is formed by forming the ultraviolet-shielding plate described previously into the shape of a quadrangle and curving it into a U-shape. The insertion holes 66 into which the fixing screws 67 are inserted are provided in both end parts of the gutter-like cover member 101 in a longitudinal direction in which the cross section thereof continues, at both sides in a direction intersecting the longitudinal direction.

In the lighting and air cleaning device 100 according to the seventh embodiment configured as described above, as shown in FIG. 21, a range from an area below the gutter-like cover member 101 to areas on both sides thereof becomes a visible light ray emission region RZ, and a space portion that is open above the gutter-like cover member 101 becomes a ultraviolet ray emission region UVZ. Then, a space portion provided between the gutter-like cover member 101 and the ultraviolet lamp 5 forms an air passage 102, and a space portion provided between the gutter-like cover member 101 and the pair of attachment rings 63, 63 forms exit and entrance ports 103.

The effects of the lighting and air cleaning device 100 configured as described above are as follows. Since the lighting and air cleaning device 100 has the pair of ferrule adaptors 62, 62 at both ends of the ultraviolet lamp 5, in a hospital, a food factory, and the like, as with a general ultraviolet tube, for example, the lighting and air cleaning device 100 can be used by being mounted to an illumination device arranged in a predetermined position of a ceiling, a wall or the like.

When electric power is fed to the lighting and air cleaning device 100, and thus the ultraviolet lamp 5 is turned on, ultraviolet rays and visible light rays are emitted from the electromagnetic wave emission portion in various directions. By the ultraviolet rays emitted from the electromagnetic wave emission portion, bacteria, viruses, and the like floating in the air within the air passage 102 formed in the gutter-like cover member 101 are killed or sterilized, and thus the air is cleaned. Here, since the surrounding air is warmed by heat generated by the ultraviolet lamp 5 and becomes light, heavy air outside the gutter-like cover member 101 enters the air passage 102 through the exit and entrance ports 103 and the opening portion arranged above, and instead, the air that has become light is discharged to the outside through the opening portion and the exit and entrance ports 69. In this way, natural convection occurs in which internal and external air flows through the air passage 102 within the lighting and air cleaning device 100, and thus sterilization by ultraviolet rays on the air passing through the air passage 102 is continuously performed.

Among the ultraviolet rays emitted from the electromagnetic wave emission portion, ultraviolet rays emitted upward through the upper opening portion of the gutter-like cover member 101 and irradiated to the ultraviolet ray emission region UVZ directly act on bacteria and viruses floating in the air within the ultraviolet ray emission region UVZ, and can kill or sterilize them. In this way, it is also possible to kill or sterilize bacteria, viruses, and the like floating in the air around the lighting and air cleaning device 100, and to clean the air within a building, and thus sterilization effects by ultraviolet rays can be effectively exerted. Here, since the lighting and air cleaning device 100 is installed in a position higher than the head of human, the ultraviolet rays emitted upward from the electromagnetic wave emission portion are not irradiated directly to the human, so that there is no fear that the human is affected by the ultraviolet rays.

At the same time, among the ultraviolet rays and the visible light rays emitted from the electromagnetic wave emission portion, the ultraviolet rays emitted downward to sideways are blocked or absorbed by the gutter-like cover member 101, whereas the visible light rays pass through the gutter-like cover member 101 and are irradiated to the visible light ray emission region RZ. Then, by the visible light rays irradiated to the visible light ray emission region RZ, a lower portion and side portions within the building are mainly illuminated, and the surrounding area thereof can be illuminated. Here, since the electromagnetic waves irradiated to the visible light ray emission region RZ are not ultraviolet rays but visible light rays, a human is not affected by the visible light rays, and the illumination functions of the lighting and air cleaning device 100 as an illumination unit can be effectively exerted.

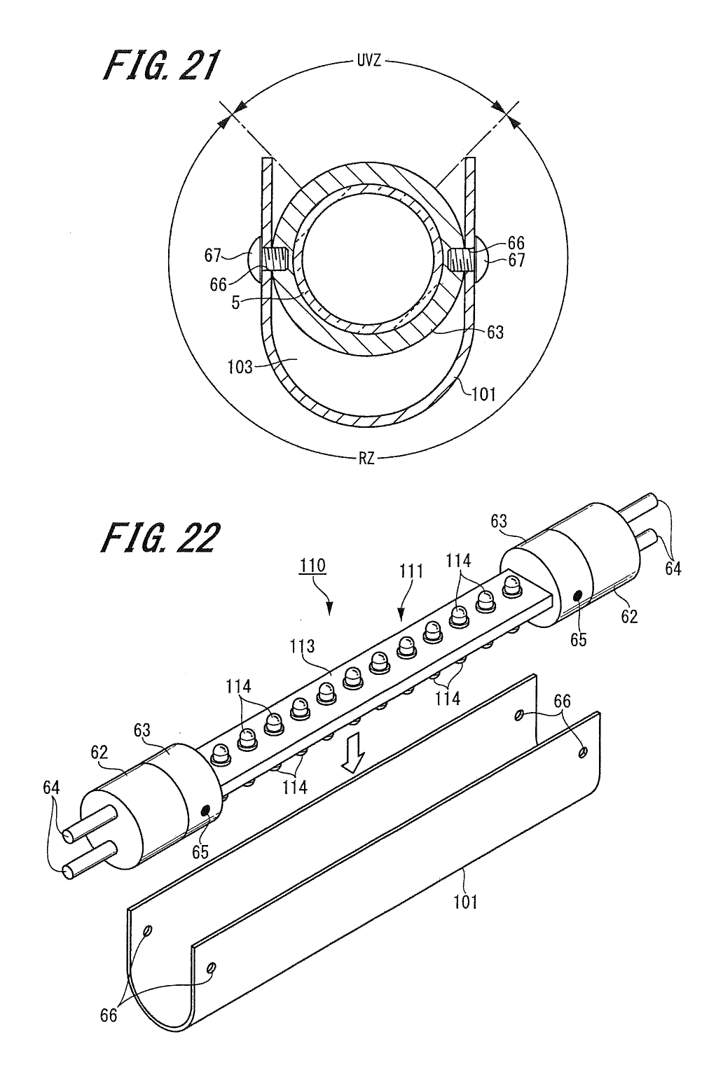

FIGS. 22 to 24 are diagrams showing the first and second variations of the seventh embodiment of the lighting and air cleaning device according to the present invention. A lighting and air cleaning device 110 as the first variation of the seventh embodiment differs from the lighting and air cleaning device 100 described above in the structure of the electromagnetic wave generation source. Hence, here, an electromagnetic wave generation source 111 will be described, and the pair of ferrule adaptors 62, 62, the pair of attachment rings 63, 63, and the gutter-like cover member 101, other than the electromagnetic wave generation source 111, are identified with like symbols, and overlapped description will not be repeated.

As shown in FIG. 22, a diode light emission member 111 indicates a second specific example of the ultraviolet generation source, and includes a long substrate 113, and a plurality of ultraviolet generation light emitting diodes 114 mounted on both surfaces of the substrate 113. The ferrule adaptors 62 are individually fixed to both ends of the substrate 113 in the longitudinal direction, the attachment rings 63 are individually attached to the ferrule adaptors 62, and they are integrally configured. On both surfaces of the substrate 113, the ultraviolet generation light emitting diodes 114, 114 are mounted so as to be spaced an appropriate distance apart. In this embodiment, the light emitting diodes 114 are arranged in one row on each of the front and rear surfaces, however, they may be arranged in two or more rows or may be arranged randomly.

On the substrate 113, a wiring circuit that electrically connects the light emitting diodes 114 and the ferrule adaptors 62 fixed at both ends is provided. The attachment rings 63 are arranged on the inside of the ferrule adaptors 62, and the gutter-like cover member 101 is attached to the side of the light source by the fixing screws 67 screwed into screw holes 65 provided in the attachment rings 63.

With the lighting and air cleaning device 110 configured as described above, it is also possible to obtain the same effects as the lighting and air cleaning device 100 described above or the like. Among the ultraviolet rays emitted from the electromagnetic wave emission portion formed of the light emitting diodes 114, ultraviolet rays emitted upward through the upper opening portion of the gutter-like cover member 101 and irradiated to the ultraviolet ray emission region UVZ act directly on bacteria and viruses floating in the air within the ultraviolet ray emission region UVZ, and can kill or sterilize them. In this way, it is possible to kill or sterilize bacteria, viruses, and the like floating in the air around the lighting and air cleaning device 110, and to clean the air within a building, and sterilization effects by ultraviolet rays can be effectively exerted.

On the other hand, among the ultraviolet rays and the visible light rays emitted from the electromagnetic wave emission portion, the ultraviolet rays emitted downward to sideways are blocked or absorbed by the gutter-like cover member 101, whereas the visible light rays and the visible light rays resulting from excitation and light emission of a light-emitting material pass through the gutter-like cover member 101 and are irradiated to the visible light ray emission region RZ. Then, by the visible light rays irradiated to the visible light ray emission region RZ, a lower portion and side portions within the building are mainly illuminated, and the surrounding area thereof can be illuminated. Here, since the electromagnetic waves irradiated to the visible light ray emission region RZ are visible light rays, a human is not affected by the visible light rays, and the illumination functions of the illumination unit can be exerted.

In particular, in this embodiment, since the ultraviolet generation source is formed of the plurality of ultraviolet generation light emitting diodes 114, as compared with the case where the ultraviolet lamp 5 is used, it is possible to greatly reduce the power consumption. About 50% of the emission from the ultraviolet light emitting diodes 114 is ultraviolet rays, and the rest is visible light rays, heat energy, and the like. Hence, when the ultraviolet light emitting diodes 114 are used as the light source, since the proportion that ultraviolet rays are emitted is increased, it is possible to increase the efficiency of killing or sterilizing bacteria, viruses, and the like with the ultraviolet rays.

FIG. 24 is a diagram showing the second variation of the seventh embodiment. A lighting and air cleaning device 120 of the second variation of the seventh embodiment differs from the lighting and air cleaning device 110 according to the first variation in the structure of the electromagnetic wave generation source and the method of attaching it. Hence, here, the structure of the electromagnetic wave generation source and the method of attaching it will be described, like parts such as the gutter-like cover member 101, etc. are identified with like symbols, and overlapped description will not be repeated.

As shown in FIG. 24, a diode light emission member 121 that is an ultraviolet generation source includes the long substrate 113, the plurality of ultraviolet generation light emitting diodes 114 mounted on one surface of the substrate 113, and a plurality of visible light ray generation light emitting diodes 122 mounted on the other surface of the substrate 113. The ferrule adaptors 62 are individually fixed to both ends of the substrate 113 in the longitudinal direction, the attachment rings 63 are individually attached to the ferrule adaptors 62, and they are integrally configured. In this embodiment, the ultraviolet generation light emitting diodes 114 and the visible light ray generation light emitting diodes 122 are each arranged in one row, however, they may be naturally arranged in two or more rows, or may be arranged randomly.

The diode light emission member 121 is arranged such that the ultraviolet generation light emitting diodes 114 are opposite the recess surface side of the gutter-like cover member 101 which is curved into a U-shape and the visible light ray generation light emitting diodes 122 face the opening portion side. The gutter-like cover member 101 is screwed to the attachment rings 63 with the fixing screws 67, and thus the lighting and air cleaning device 120 is configured.

With the lighting and air cleaning device 120 configured as described above, it is also possible to obtain the same effects as the lighting and air cleaning device 110 described above or the like. The lighting and air cleaning device 120 is mounted to an illumination unit installed on a ceiling or the like with the visible light ray generation light emitting diodes 122 pointing downward. When the lighting and air cleaning device 120 is energized, the ultraviolet generation light emitting diodes 114 mounted on the upper surface of the substrate 113 emit ultraviolet rays and the like in an upward direction, and the visible light ray generation light emitting diodes 122 mounted on the lower surface of the substrate 113 emit visible light rays in a downward direction.

The ultraviolet rays emitted from the light emitting diodes 114 of the diode light emission member 121 are directly irradiated to bacteria and viruses floating in the air within the air passage 102 above it, and thus the bacteria and the like are directly killed or sterilized with the ultraviolet rays. The air within the air passage 102 is warmed by heat generated from the diode light emission member 121 and becomes light, and is moved in the air passage 102 and is discharged to the outside. On the other hand, instead, heavy air enters the air passage 102. By natural convection generated here, bacteria, viruses and the like floating in the air around the lighting and air cleaning device 120 are killed or sterilized, and, by repeating this action, it is possible to clean the air within a building.

On the other hand, the visible light rays emitted from the light emitting diodes 122 of the diode light emission member 121 are irradiated directly to the visible light ray emission region through the opening portion of the gutter-like cover member 101. The visible light rays irradiated to the visible light ray emission region mainly illuminate a lower area and side areas within a building, and can brightly illuminate the surrounding area. Here, since the electromagnetic waves irradiated to the visible light ray emission region are visible light rays, a human is not affected by the visible light rays, and the illumination functions of the lighting and air cleaning device 120 as an illumination unit can be exerted.

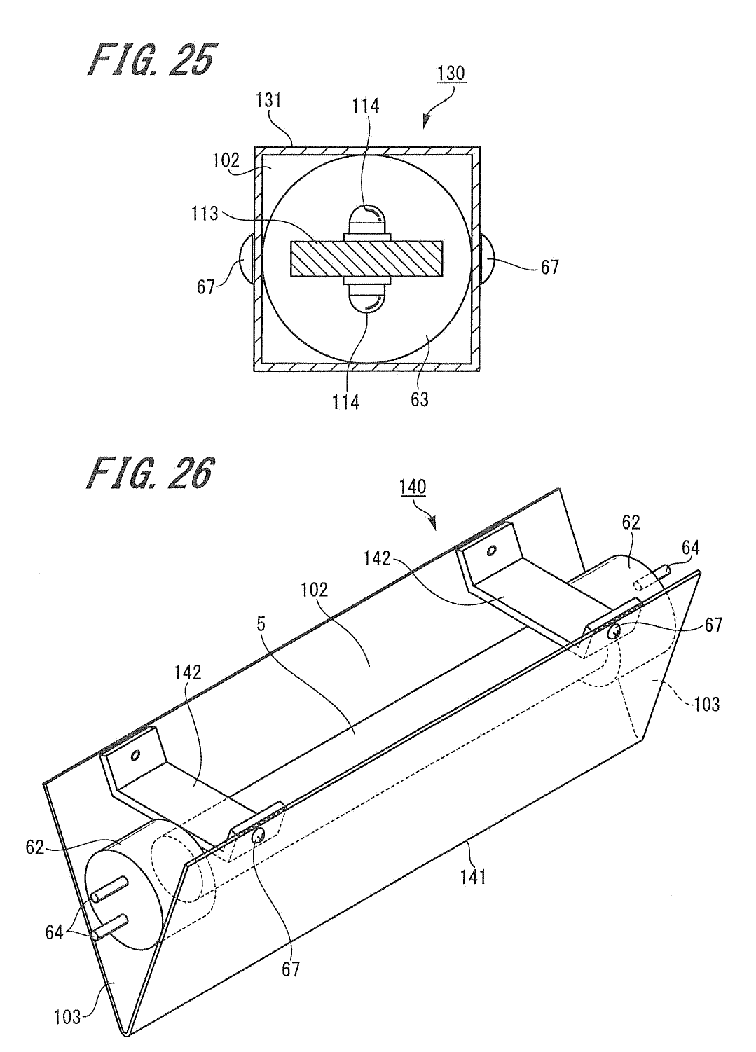

FIG. 25 is a diagram showing a third variation of the seventh embodiment. A lighting and air cleaning device 130 of the third variation of the seventh embodiment differs from the lighting and air cleaning device 110 according to the first variation in a cover tube member 131. Hence, here, the cover tube member 131 will be described, like parts such as the electromagnetic wave generation source 111, etc. are identified with like symbols, and overlapped description will not be repeated.

As shown in FIG. 25, the cover tube member 131 is formed of an ultraviolet-shielding resin member made of ultraviolet non-transmissive and visible-light transmissive resin, in the shape of a square tube whose cross section is quadrangular. The other parts of the configuration are the same as those in the embodiments described above. With this configuration, it is also possible to obtain the same effects as in the embodiments described above.

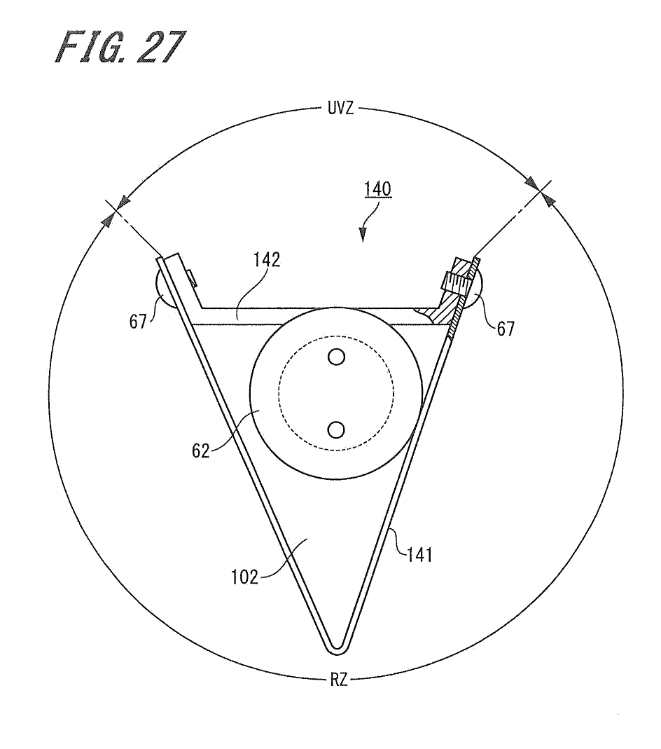

FIGS. 26 and 27 are diagrams showing the fourth variation of the seventh embodiment. A lighting and air cleaning device 140 of the fourth variation of the seventh embodiment differs from the lighting and air cleaning device 100 according to the seventh embodiment described above in a gutter-like cover member 141 and a fixing arm 142. Hence, here, the gutter-like cover member 141 and the fixing arm 142 will be described, like parts such as the ultraviolet lamp 5, etc. are identified with like symbols, and overlapped description will not be repeated.

As shown in FIGS. 26 and 27, the gutter-like cover member 141 is formed by bending, in a V-shape, an ultraviolet non-transmissive and visible-light transmissive ultraviolet-shielding plate. The fixing arms 142 holding the opening side of the gutter-like cover member 141 at a predetermined interval intervene at two places on the opening side of the gutter-like cover member 141. The two fixing arms 142 are fixed to end surface portions on the inside of the pair of ferrule adaptors 62, 62 attached to both ends of the ultraviolet lamp 5 in the axial direction. Both end portions of the fixing arm 142 in the longitudinal direction are bent at about the same angle as the angle at which the gutter-like cover member 141 is bent, and thus the contact area of the fixing arm 142 with the gutter-like cover member 141 is increased, and screwing with the fixing screws 67 is facilitated. The other parts of the configuration are the same as in the embodiments described above. With this configuration, it is also possible to obtain the same effects as in the embodiments described above.

### Eighth Embodiment

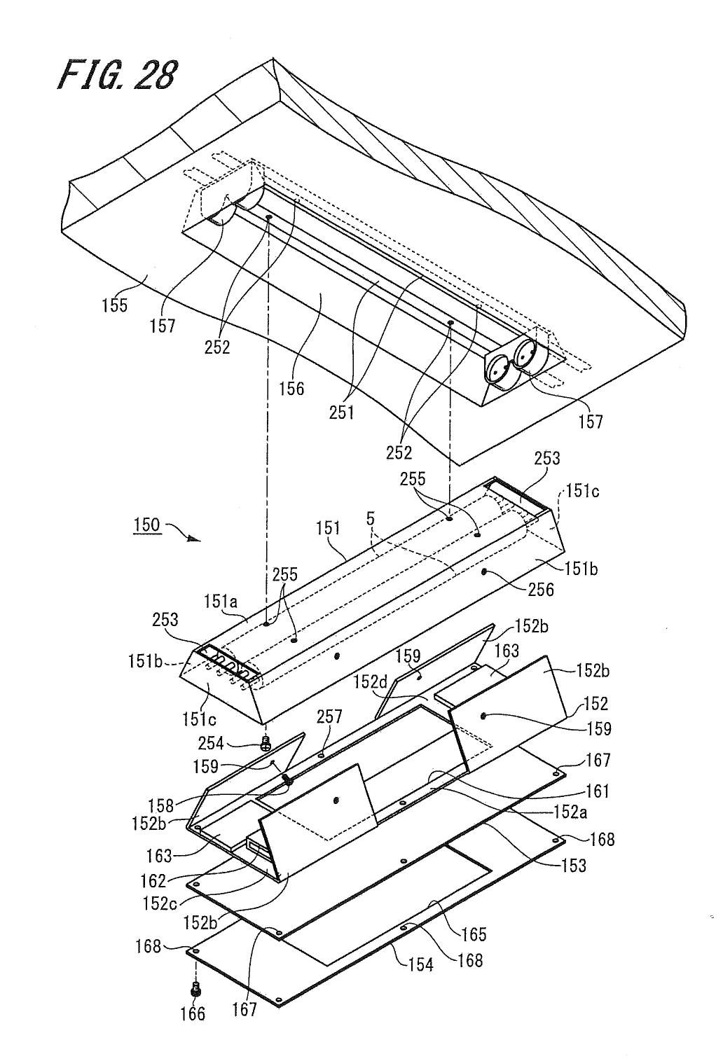

FIGS. 28 to 30 are diagrams showing the eighth embodiment of the lighting and air cleaning device according to the present invention. A lighting and air cleaning device 150 according to the eighth embodiment is configured such that it is installed by being embedded in a predetermined position in a ceiling of a building or the like. The lighting and air cleaning device 150 includes an attachment frame 151, an external plate 152, an ultraviolet non-transmissive and visible-light transmissive resin plate 153, a pressing plate 154, and the ultraviolet lamp 5 that is an ultraviolet generation source.

A slit portion 156 into which the attachment frame 151 is fitted is provided in the ceiling 155 indicating a specific example of the predetermined position. The slit portion 156 has a shape corresponding to the attachment frame 151, and is formed in such a shape and a size that the entire attachment frame 151 is fit in the slit portion 156. In the ceiling surface of the slit portion 156, two fixing bars 251, 251 for fixing the lighting and air cleaning device 150 are provided (the number of fixing bars may be one, or three or more). The two fixing bars 251 are arranged parallel so as to across the ceiling 155 and lower surfaces thereof are exposed to the slit portion 156. In each of the fixing bars 251, 251, unillustrated screw holes for screwing sockets 157, 157, and two screw holes 252 for screwing the attachment frame 151 are provided. A plurality of (in this embodiment, two) ultraviolet lamps 5 are removably mounted to the pair of sockets 157 screwed to the pair of fixing bars 251.

The attachment frame 151 that is fit in the slit portion 156 has a rectangular upper surface portion 151a, first side surface portions 151b, 151b that are continuous to both sides of the upper surface portion 151a in the axial direction, and second side surface portions 151c, 151c that are continuous to both sides of the upper surface portion 151a in the longitudinal direction, and the surface opposite the upper surface portion 151a is an opening portion. The opening portion of the attachment frame 151 is set wider than the width of the upper surface portion 151a. In both sides of the upper surface portion 151a of the attachment frame 151 in the longitudinal direction, penetration holes 253, into which the sockets 157 fixed to the pair of fixing bars 251, 251 in the slit portion 156 are inserted, are provided.

Furthermore, in the upper surface portion 151a of the attachment frame 151, insertion holes 255 for screwing, with fixing screws 254, the attachment frame 151 to the pair of fixing bars 251, 251 of the slit portion 156 are provided in four places. In each of the first side surface portions 151b, 151b of the attachment frame 151, screw holes 256 for screwing the external plate 152 with fixing screws 158 are provided in two places.

The external plate 152 is removably attached, with the fixing screws 158, to the attachment frame 151 fit in the slit portion 156. The external plate 152 has a flat surface portion 152a that has substantially the same size corresponding to the opening portion of the attachment frame 151, and four attachment parts 152b, 152b that are provided continuous to four places of the flat surface portion 152a. The four attachment parts 152b, 152b are arranged at four places so as to be on both sides of the external plate 152 in the longitudinal direction and opposite both sides in the width direction. In each of the attachment parts 152b, an insertion hole 159, into which the screw shaft portion of the fixing screw 158 is inserted, is provided, and the screw holes 256 corresponding to these insertion holes 159 are provided in predetermined positions of the first side surface portions 151b of the attachment frame 151.

In the flat surface portion 152a of the attachment frame 151, an opening window 161 is provided from which a large part of the ultraviolet emission portions of the two ultraviolet lamps 5 mounted into the pair of the sockets 157, 157 can be exposed. On a ledge portion 152c on one side of the opening window 161 of the flat surface portion 152a in the longitudinal direction, a fan 162 for forcibly moving air and a solar panel 163 are provided. Furthermore, on a ledge portion 152d on the other side of the opening window 161 of the flat surface portion 152a in the longitudinal direction, a plurality of solar panels 163 is provided.

The fan 162 is provided to forcibly send air into an air passage 164 formed on the inside of the external plate 152 and move the air. The solar panel 163 produces electrical energy for driving the fan 162. The electrical energy produced by the solar panel 163 can be directly used for driving the fan 162 or can be temporarily stored in a storage battery or the like and be taken and used as necessary. This solar panel 163 can produce electrical energy based on electromagnetic waves emitted from the ultraviolet lamps 5.

Outside the opening window 161 provided in the flat surface portion 152a of the attachment frame 151, an ultraviolet non-transmissive and visible-light transmissive resin plate (ultraviolet-shielding plate) 153 is arranged so as to cover the entire surface of the opening window 161. Needless to say, instead of the ultraviolet non-transmissive and visible-light transmissive resin plate 153, an ultraviolet transmissive and visible-light transmissive plate or film that transmits ultraviolet rays and that transmits visible light rays emitted by exciting the light-emitting material with the ultraviolet rays, or the one obtained by coating the plate or film with an ultraviolet non-transmissive and visible-light transmissive fluorine resin can be used. The pressing plate 154 is arranged on the outside of the ultraviolet-shielding plate 153.

In the pressing plate 154, an opening window 165 is provided that has about the same size and shape as the opening window 161. In the ultraviolet-shielding plate 153, insertion holes 167, into which the screw shaft portions of fixing screws 166 are inserted, are provided, and likewise, in the pressing plate 154, insertion holes 168, into which the screw shaft portions of the fixing screws 166 are inserted, are provided (in the present embodiment, six for each). In the positions of the flat surface portion 152a corresponding to the insertion holes 167 and the insertion holes 168, screw holes 257 are provided into which the screw shaft portions of the fixing screws 166 are screwed. Although not shown, in the ultraviolet-shielding plate 153 and the pressing plate 154, at positions overlapping the fan 162 and the solar panel 163, air holes, through which air is passed, and fitting holes, in which the fan 162 and the solar panel 163 are fitted together, may be provided.

The lighting and air cleaning device 150 configured as described above can be assembled, for example as follows. First, the pair of sockets 157, 157 is screwed into the slit portion 156 provided in the ceiling 155. Then, the pair of sockets 157, 157 is inserted through the penetration holes 253, 253 of the attachment frame 151, and the attachment frame 151 is fit in the slit portion 156. Then, the screw shaft portions of the fixing screws 254 are inserted through the insertion holes 255 and are screwed into the screw holes 252 of the fixing bars 251, and thus the four fixing screws 254 are used to fix the attachment frame 151 to the slit portion 156 of the ceiling 155.

Then, the two ultraviolet lamps 5 are attached to the pair of sockets 157, 157 fixed within the slit portion 156. Then, the external plate 152 is attached to the attachment frame 151, and a plurality of fixing screws 158 are used to tighten and fix the external plate 152 to the attachment frame 151. In this case, the fan 162 and the solar panel 163 are previously attached to the external plate 152, and the opening window 161 is held open. In this way, a hand can be inserted through the opening window 161, and, in this state, the fixing screws 158 are tightened, and thus the assembly operation of the external plate 152 can be performed.

Then, the ultraviolet-shielding plate 153 is set on the outside of the opening window 161 of the external plate 152, and the pressing plate 154 is set on the outer edge of the ultraviolet-shielding plate 153. Thereafter, a plurality of fixing screws 166 is screwed to tighten the ultraviolet-shielding plate 153 with the pressing plate 154. In this way, the assembly operation of the lighting and air cleaning device 150 is completed.

With the lighting and air cleaning device 150 configured as described above, it is possible to obtain the same effects as the lighting and air cleaning device 1 described previously or the like. When the ultraviolet lamps 5 of the lighting and air cleaning device 150 are turned on, ultraviolet rays and visible light rays are emitted from the electromagnetic wave emission portion in various directions. The ultraviolet rays emitted from the electromagnetic wave emission portion kill or sterilize bacteria, viruses, and the like floating in the air around the electromagnetic wave emission portion within the space enclosed by the attachment frame 151 and the external plate 152, and thus the air is cleaned.

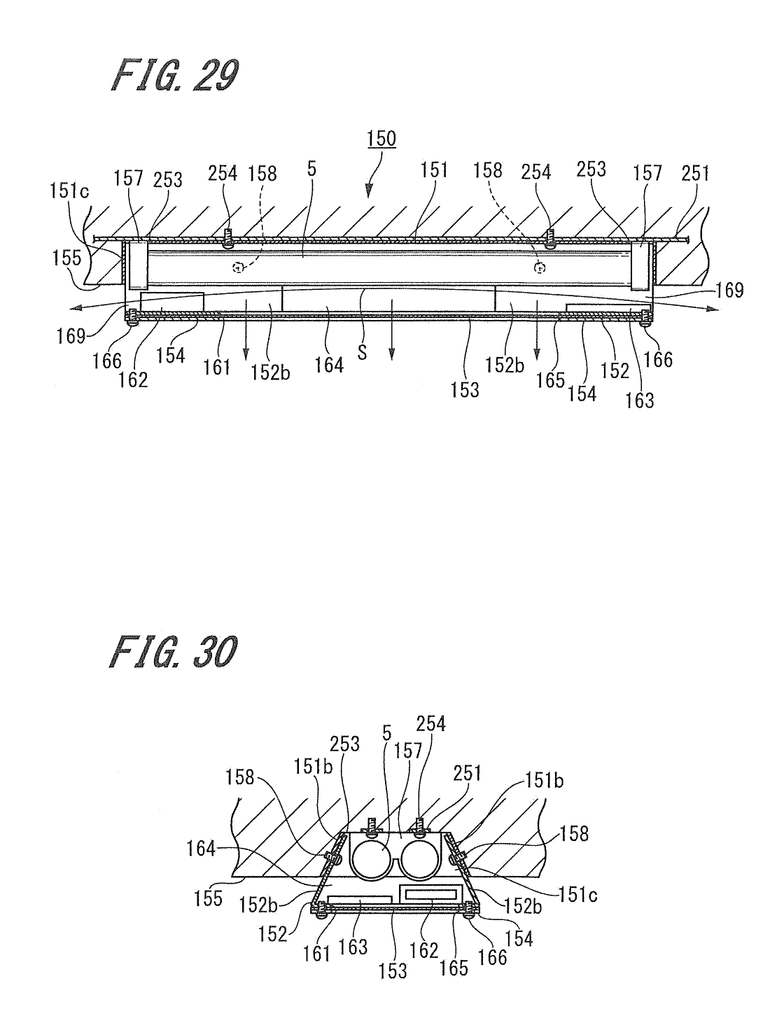

Furthermore, as shown in FIGS. 29 and 30, the surrounding air is warmed by the heat energy generated when the ultraviolet lamps 5 emit ultraviolet rays and the like, and the natural convection of air indicated by an arrow S occurs. Consequently, the internal air is warmed to become light, and the light air is pushed, by external heavy and cold air, through exit and entrance ports 169 on the sides of the air passage 164, to the outside. Thus, the air is convected in a horizontal direction, and bacteria, viruses, and the like floating in newly supplied air are likewise killed or sterilized by the ultraviolet rays emitted from the ultraviolet lamps 5. By repeating the convection of air and the ultraviolet sterilization described above, it is possible to continuously clean the air within the building 33. In this case, the fan 162 is driven to forcibly move the air, and thus it is possible to increase the range of ultraviolet sterilization and the efficiency of cleaning of the air within the building.

Among the ultraviolet rays and the visible light rays emitted from the ultraviolet lamps 5, ultraviolet rays moving downward are blocked or absorbed by the ultraviolet-shielding plate 153 attached to the flat surface portion 152a of the external plate 152, whereas the ultraviolet rays passes through the ultraviolet non-transmissive and visible-light transmissive ultraviolet-shielding plate 153 that transmits ultraviolet rays and that transmits visible light rays emitted by exciting, with the ultraviolet rays, the light-emitting material. The ultraviolet-shielding plate 153 may be the one coated with ultraviolet transmissive and visible-light transmissive fluorine resin. The visible light rays irradiated to the visible light ray emission region formed of the opening window 161 of the external plate 152 and the opening window 165 of the pressing plate 154 mainly illuminate a lower area within the building, and thus it is possible to brightly illuminate the surrounding area. Here, since the electromagnetic waves irradiated to the visible light ray emission region are not ultraviolet rays but visible light rays, a human is not affected by the visible light rays, and the illumination functions of the lighting and air cleaning device 150 as an illumination unit can be exerted.

### Ninth Embodiment

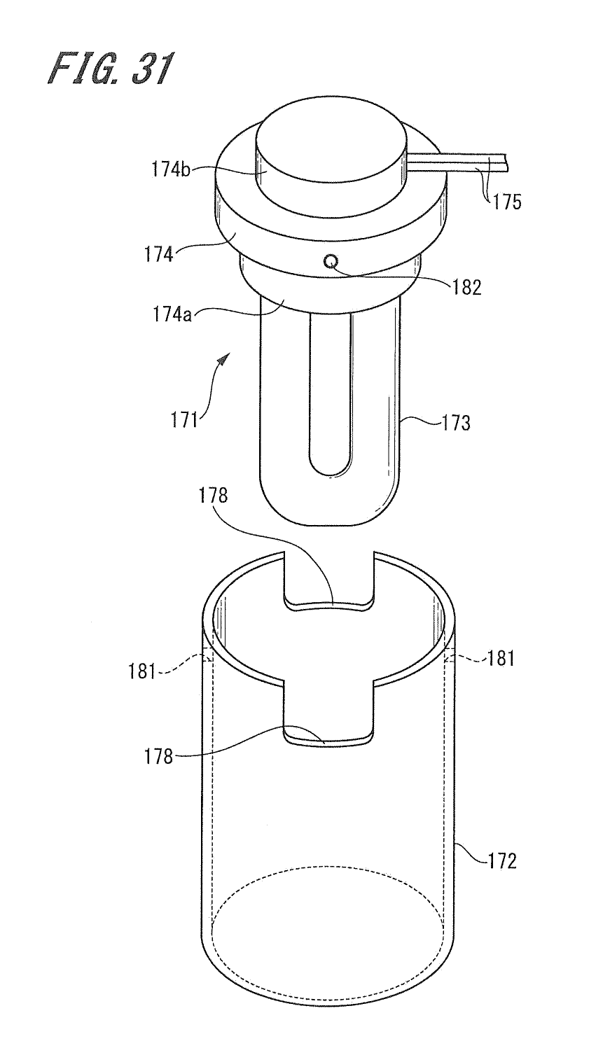

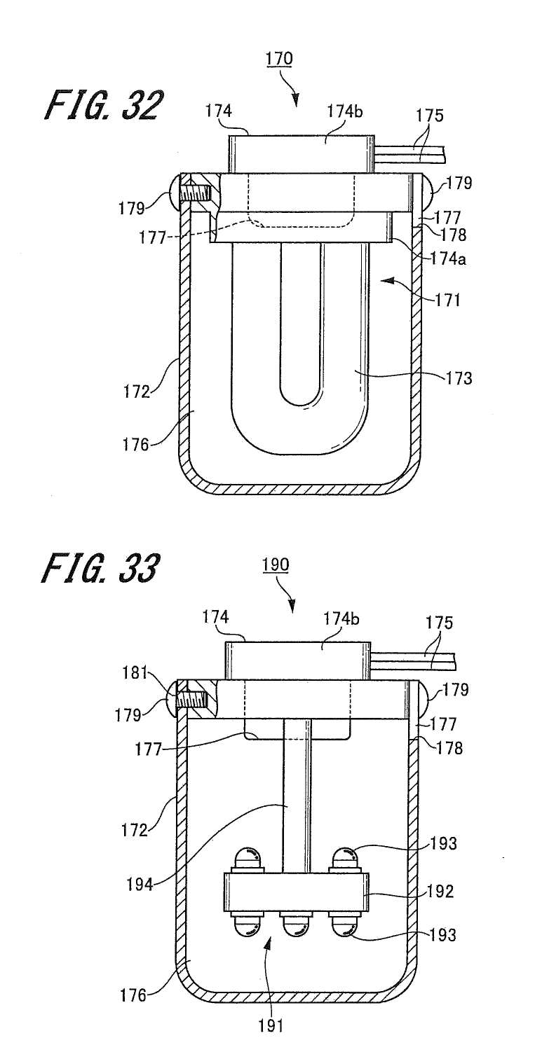

FIGS. 31 to 35 are diagrams showing the ninth embodiment and its variations of the lighting and air cleaning device according to the present invention. A lighting and air cleaning device 170 according to the ninth embodiment, shown in FIGS. 31 and 32, includes an ultraviolet lamp 171 that is an electromagnetic wave generation source, and an one-end-opened cup 172 indicating a sixth specific example of the ultraviolet-shielding resin member.

The ultraviolet lamp 171 is formed of a U-shaped glass tube 173, and a ferrule member 174 that closes an end portion on the opening side of the glass tube 173. The ferrule member 174 is formed of a disk-shaped member that closes the opening portion of the one-end-opened cup 172. On one end surface of the disk-shaped member, a glass tube support portion 174a is provided that supports the opening end of the glass tube 173, and, on the other end surface, a lamp fixing portion 174b is provided from which a lead wire 175 is drawn. In the glass tube support portion 174a, a pair of electrodes is provided that are inserted into the glass tube 173. The lamp fixing portion 174b is a portion for attaching the lighting and air cleaning device 170 to a predetermined portion of a wall, a floor or the like. An unillustrated fixing screw, an adhesive or other means is used to fix the lamp fixing portion 174b to the predetermined position, and thus it is possible to attach the lighting and air cleaning device 170 to a desired attachment position.

The one-end-opened cup 172 is formed of a bottomed tube member with only one end opened, and can be formed of any one of the ultraviolet non-transmissive and visible-light transmissive plate or film described previously and a fluorine resin having a thickness of about 5 mm. The thickness of the tube member is set at about 1.5 mm to 3.0 mm, using the ultraviolet transmissive and visible-light transmissive fluorine resin material, and at least one of its inner and outer surfaces is coated with a fluorescent substance and the surface thereof can also be coated with the ultraviolet transmissive and visible-light transmissive fluorine resin. In the edge portion of the one-end-opened cup 172 on the opening side, notches that form exit and entrance ports 177 of an air passage 176 are provided in two places. Furthermore, in the edge portion of the one-end-opened cup 172 on the opening side, insertion holes 181 into which the ferrule member 174 is screwed with fixing screws 179 are provided in two places. In the outer circumference of the ferrule member 174, screw holes 182, into which the screw shaft portions of the fixing screws 179 are screwed, are provided corresponding to the two insertion holes 181.

With the lighting and air cleaning device 170 configured as described above, it is possible to obtain the same effects as the lighting and air cleaning device 1 described previously or the like. Specifically, when the ultraviolet lamps 171 of the lighting and air cleaning device 170 are turned on, ultraviolet rays and visible light rays are emitted from the electromagnetic wave emission portion in various directions. The ultraviolet rays emitted from the electromagnetic wave emission portion kill or sterilize bacteria, viruses, and the like floating in the air around the electromagnetic wave emission portion within the air passage 176 enclosed by the one-end-opened cup 172, and thus the air is cleaned.

Furthermore, as shown in FIGS. 31 and 32, the surrounding air is warmed by the heat energy generated when the ultraviolet lamp 171 emits ultraviolet rays and the like to thereby become light, and rises in the air passage 176 within the one-end-opened cup 172. Then, the air is discharged to the outside through one of the exit and entrance ports 177 of the air passage 176 formed of the notches 178 provided in the opening side edge portion of the one-end-opened cup 172, and, instead, external cold and heavy air enters the one-end-opened cup 172 through the other exit and entrance port 177. Thus, the air is naturally convected in an up/down direction, and bacteria, viruses and the like floating in newly supplied air are likewise killed or sterilized by the ultraviolet rays. By repeating the convection of air and the ultraviolet sterilization described above, it is possible to continuously clean the air.

Among the ultraviolet rays and the visible light rays emitted from the ultraviolet lamp 171, ultraviolet rays moving downward and sideway are blocked or absorbed by the one-end-opened cup 172, whereas the visible light rays pass through the one-end-opened cup 172. The visible light rays passing through the one-end-opened cup 172 and visible light rays emitted by exciting the light-emitting material with ultraviolet rays illuminate the surrounding of the lighting and air cleaning device 170, and brightly illuminate the surrounding area. Here, since the ultraviolet rays are blocked or absorbed by the one-end-opened cup 172, and only the visible light rays are irradiated to the visible light ray emission region, a human is not affected by the visible light rays, and the illumination functions of the illumination unit can be exerted.

FIG. 33 shows a first variation of the ninth embodiment according to the lighting and air cleaning device of the present invention. A lighting and air cleaning device 190 of the first variation of the ninth embodiment differs from the lighting and air cleaning device 170 described above in only a diode light emission member 191 that is an ultraviolet generation source. Hence, here, the diode light emission member 191 will be described, like parts such as the one-end-opened cup 172, the ferrule member 174, etc. are identified with like symbols, and overlapped description will not be repeated.

As shown in FIG. 33, the diode light emission member 191 includes a disk-shaped substrate 192, a plurality of ultraviolet generation light emitting diodes 193 mounted on both surfaces of the substrate 192, and a stand 194 that holds the substrate 192 at a predetermined height. The stand 194 is provided to stand in the center on one surface of the ferrule member 174, and the ultraviolet generation light emitting diodes 193 mounted on the substrate 192 through the lead wire inserted into the stand 194 are connected to the lead wire 175 within the ferrule member 174. With the semiconductor device 190 configured as described above, it is also possible to obtain the same effects as the lighting and air cleaning device 170.

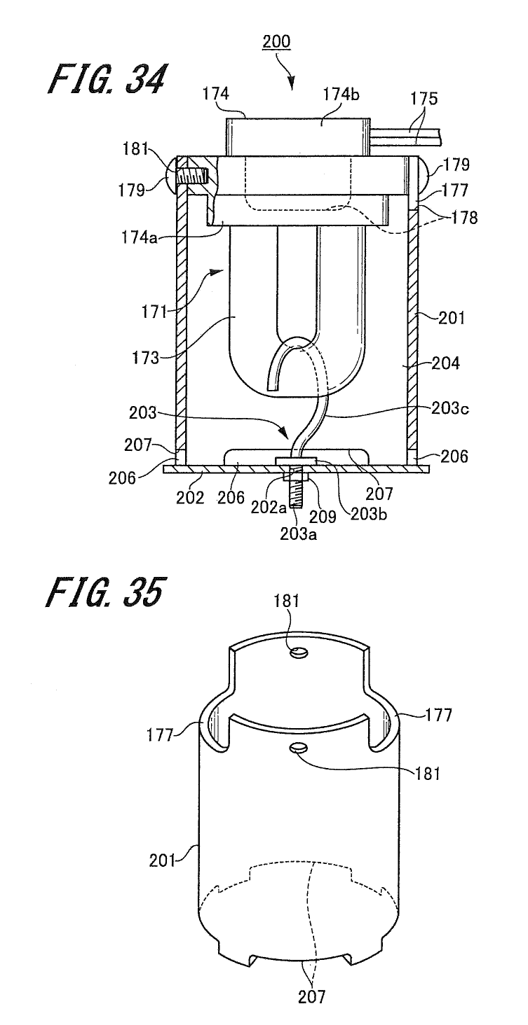

FIG. 34 is a diagram showing a second variation of the ninth embodiment according to the lighting and air cleaning device of the present invention. A lighting and air cleaning device 200 of the second variation of the ninth embodiment differs from the lighting and air cleaning device 170 described above in a both-end-opened cup 201 that is an ultraviolet-shielding resin member, its lid member 202, and its hanging hook 203. Hence, here, the both-end-opened cup 201, the lid member 202, and the hanging hook 203 will be described, like parts such as the ultraviolet lamp 171, etc. are identified with like symbols, and overlapped description will not be repeated.

As shown in FIG. 35, the both-end-opened cup 201 is formed of a both-end-opened tube member that is open to both ends in the axial direction. This both-end-opened cup 201 can be formed of any one of the ultraviolet non-transmissive and visible-light transmissive resin plate or resin film described previously, the ultraviolet transmissive and visible-light transmissive fluorine resin having a thickness of about 5 mm, and the ultraviolet transmissive and visible-light transmissive fluorine resin material in which its thickness is set at about 1.5 mm to 3.0 mm and in which at least one of its inner and outer surfaces is coated with a light-emitting material and the surface thereof is coated with the ultraviolet transmissive and visible-light transmissive fluorine resin. In an edge portion on one opening side of the both-end-opened cup 201, first notches 178 that form a first exit and entrance port 205 mainly functioning as an exit of an air passage 204 are provided in two places. In the edge portion of the both-end opened cup 201 on the side of the first notches 178, the insertion holes 181 for screwing the ferrule member 174 with the fixing screws 179 are provided in two places. In an edge portion on the other opening side of the both-end-opened cup 201, second notches 207 that form a second exit and entrance port 206 mainly functioning as an entrance of the air passage 204 are provided in four places.

The opening portion of the both-end-opened cup 201 on the side of the second notches 207 is closed by the lid member 202. The lid member 202 is formed of a disk-shaped plate member having a larger diameter than the outer diameter of the both-end-opened cup 201, and can be formed of the same material as the both-end-opened cup 201. The lid member 202 is supported by the hanging hook 203. Hence, in the center portion of the lid member 202, an insertion hole 202a through which a screw shaft portion 203a of the hanging hook 203 is inserted is provided. In one end of the screw shaft portion 203a of the hanging hook 203 in the axial direction, a flange portion 203b is provided, and on the opposite side to the screw shaft portion 203a of the flange portion 203b, an arc-shaped curved hook portion 203c is integrally provided.

The hook portion 203c of the hanging hook 203 is formed such that its radius of curvature is appropriately larger than the diameter of the glass tube 173 of the ultraviolet lamp 171, and such that it can be hooked on the curved portion of the glass tube 173. A nut 209 is screwed over the screw shaft portion 203a of the hanging hook 203. The nut 209 screwed over the screw shaft portion 203a supports the lid member 202 that closes the opening portion of the both-end-opened cup 201 on the side of the second notches 207.

With the lighting and air cleaning device 200 configured as described above, it is also possible to obtain the same effects as the lighting and air cleaning device 170 described previously or the like. Specifically, when the ultraviolet lamp 171 of the lighting and air cleaning device 200 is turned on, ultraviolet rays and visible light rays are emitted from the electromagnetic wave emission portion in various directions. The ultraviolet rays emitted from the electromagnetic wave emission portion kill or sterilize bacteria, viruses, and the like floating in the air around the electromagnetic wave emission portion within the air passage 204 enclosed by the both-end-opened cup 201 and the lid member 202, and thus the air is cleaned.

Furthermore, as shown in FIG. 34, the surrounding air is warmed by the heat energy generated when the ultraviolet lamp 171 emits ultraviolet rays and the like to thereby become light, and rises in the air passage 204 within the both-end-opened cup 201. Then, the air that has been sterilized with the ultraviolet rays and that has been warmed to become light is discharged to the outside through the first exit and entrance port 177 formed of the first notches 178 provided in the edge portion of the upper opening portion of the both-end-opened cup 201. Instead, external cold and heavy air enters the air passage 204 through the second exit and entrance port 206 formed of the second notches 207 provided in the edge portion of the lower opening portion of the both-end-opened cup 201. Thus, the air is naturally convected in an up/down direction, and bacteria, viruses, and the like floating in newly supplied air are likewise killed or sterilized by the ultraviolet rays emitted from the ultraviolet lamp 171. By repeating the convection of air and the ultraviolet sterilization described above, it is possible to continuously clean the air.

Among the ultraviolet rays and the visible light rays emitted from the ultraviolet lamp 171, ultraviolet rays moving downward and sideway are blocked or absorbed by the both-end-opened cup 201 and the lid member 202, whereas the visible light rays pass through the both-end-opened cup 201 and the lid member 202. The visible light rays passing through the both-end-opened cup 201 and the like illuminate the surrounding of the lighting and air cleaning device 200 to brightly illuminate the surrounding area. Here, since the ultraviolet rays are blocked or absorbed by the both-end-opened cup 201 and the lid member 202, and the visible light rays are irradiated to the visible light ray emission region, a human is not affected by the visible light rays, and the visible light rays emitted by exciting a light-emitting material with ultraviolet rays are irradiated as illumination light, and thus the illumination functions of the lighting and air cleaning device 200 as an illumination unit can be exerted.

### Tenth Embodiment

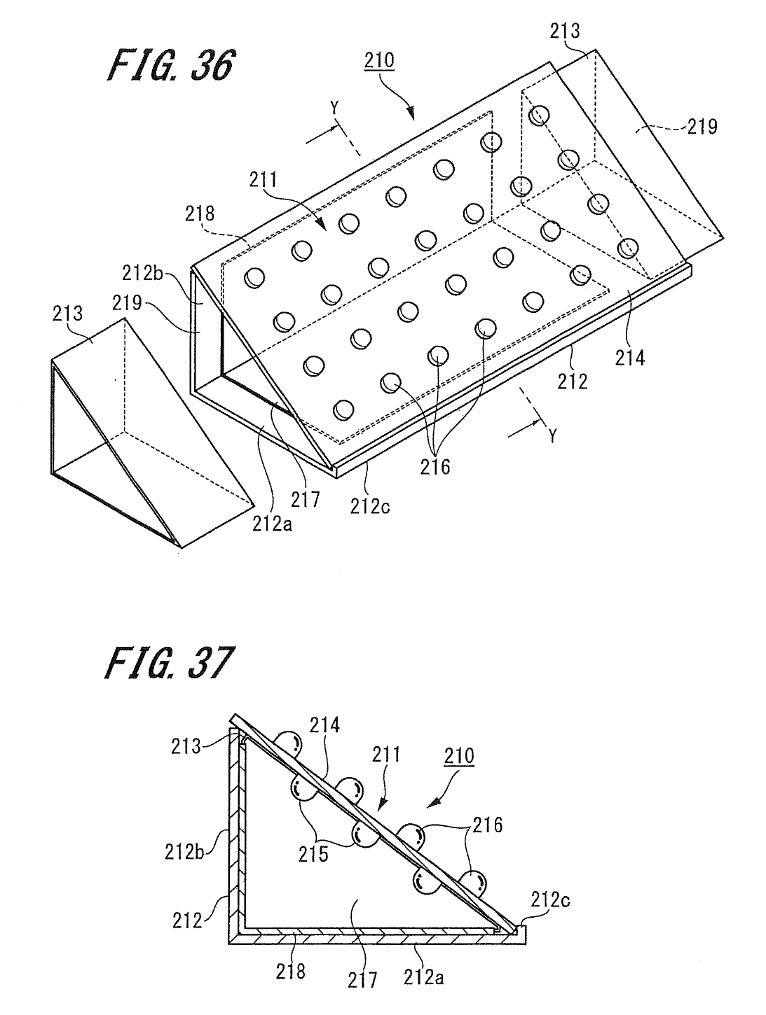

FIGS. 36 to 40 are diagrams showing the tenth embodiment and its variations of the lighting and air cleaning device according to the present invention. A lighting and air cleaning device 210 according to the tenth embodiment, shown in FIGS. 36 and 37, includes a diode light emission member 211 that is an electromagnetic wave generation source, a light emission member stand 212 that supports the diode light emission member 211 in an inclined state, and a pair of tunnel tube members 213, 213 that indicate a seventh specific example of the ultraviolet-shielding resin member. The lighting and air cleaning device 210 is configured so as to be suitable for being used while placed to stand or lie in a corner of a room, a partition, a screen or the like of a building.

The diode light emission member 211 includes a substrate 214 that is formed in a rectangular, a large number of ultraviolet generation light emitting diodes 215 that are arranged on one surface of the substrate 214, and a large number of visible light ray generation light emitting diodes 216 that are arranged on the other surface of the substrate 214. In this embodiment, the ultraviolet generation light emitting diodes 215 and the visible light ray generation light emitting diodes 216 are regularly arranged both in an up/down direction and in a left/right direction, however, naturally, they may be randomly arranged in any case. The substrate 214 is attached to the light emission member stand 212 with the ultraviolet generation light emitting diodes 215 facing inward and the visible light ray generation light emitting diodes 216 facing outward.

The light emission member stand 212 supports the diode light emission member 211 in an inclined state, has a bottom surface portion 212a having substantially the same length as the substrate 214, and a side surface portion 212b rising continuously on one side of the bottom surface portion 212a in the width direction, and is formed such that its cross section is L-shaped as a whole. In the other side of the bottom surface portion 212a in the width direction, a hook portion 212c that supports the lower end portion of the diode light emission member 211 is provided. The width of the diode light emission member 211 and the height of the side surface portion 212b of the light emission member stand 212 are set such that, with the lower end portion of the diode light emission member 211 hooked in the hook portion 212c, its upper end portion reaches the upper end portion of the side surface portion 212b. A space portion of a triangle pole shape enclosed by the diode light emission member 211 and the light emission member stand 212 forms an air passage 217 through which air moves.

On the inside of the light emission member stand 212, a reflective plate 218 that reflects ultraviolet rays emitted from the ultraviolet generation light emitting diodes 215 is preferably provided. Preferably, the reflective plate 218 is formed such that the cross section thereof is L-shaped corresponding to the shape of the light emission member stand 212, and the two surfaces thereof are supported by the bottom surface portion 212a and the side surface portion 212b of the light emission member stand 212. The tunnel tube members 213, 213 that outwardly extend the exit and entrance ports 219 of the air passage 217 are attached to both sides of the diode light emission member 211 in the longitudinal direction. In this embodiment, the tunnel tube member 213 is formed as a triangular tube member, and as its material, any one of an ultraviolet non-transmissive and visible-light transmissive resin plate or resin film and an ultraviolet transmissive and visible-light transmissive fluorine resin having a thickness of about 5 mm can be applied.

The tunnel tube members 213 may be fixed to the substrate 214 with an adhesive, or may be screwed with fixing screws. Since the tunnel tube members 213 do not cover the exit and entrance ports 219 of the air passage 217, they cannot block or absorb ultraviolet rays passing through the interior of the tunnel tube members 213, but can reduce the range of irradiation of ultraviolet rays to the outside and thereby reduce a range in which ultraviolet rays may directly enter the eyes of a human. The thickness of the tunnel tube members 213 is set at about 1.5 mm to 3.0 mm, using the ultraviolet transmissive and visible-light transmissive fluorine resin material, at least one of its inner and outer surfaces is coated with a fluorescent substance and the surface thereof is coated with the ultraviolet transmissive and visible-light transmissive fluorine resin, and thus the tunnel tube members 213 can convert ultraviolet rays into visible light rays and discharge them.

With the lighting and air cleaning device 210 configured as described above also, it is possible to obtain the same effects as the lighting and air cleaning device 1 described previously or the like. Specifically, when the lighting and air cleaning device 210 is energized, ultraviolet rays emitted from the ultraviolet generation light emitting diodes 215 mounted on one surface of the substrate 214 into the interior of the air passage 217 directly act on bacteria, viruses, and the like floating in the air within the air passage 217, and thus it is possible to kill or sterilize the bacteria and the like with the ultraviolet rays. In this way, it is possible to kill or sterilize bacteria, viruses, and the like floating in the air passing through the air passage 217, and to clean the air within a building, and thus sterilization effects by ultraviolet rays can be effectively exerted.

On the other hand, the visible light rays emitted from the visible light ray generation light emitting diodes 216 mounted on the other surface of the substrate 214 into the room illuminate the interior of the building, and can brightly illuminate the surrounding area. Here, since the electromagnetic waves emitted from the visible light ray generation light emitting diodes 216 are visible light rays, even if the visible light rays directly enter the eyes of a human, the human is not affected by the visible light rays. Moreover, even if the visible light rays are irradiated to skin, there is no possibility that the skin is affected. Hence, the lighting and air cleaning device 210 can exert the illumination functions as an illumination unit. In particular, when, as in this embodiment, a large number of ultraviolet generation light emitting diodes 215 are used as the ultraviolet generation source, as compared with the case where the ultraviolet lamp 5 is used, it is possible to greatly reduce power consumption, increase the proportion of emitting ultraviolet rays and enhance the efficiency of killing or sterilizing bacteria, viruses, and the like with ultraviolet rays.

In the embodiment shown in FIGS. 36 and 37, by eliminating the visible light ray generation light emitting diodes 216 and using only the ultraviolet generation light emitting diodes 215, and at the same time by forming the substrate 214 with the ultraviolet non-transmissive and visible-light transmissive resin plate or resin film, and causing visible light rays emitted by exciting a light-emitting material through the collision of ultraviolet rays with the light-emitting material to transmit the substrate 214, it is also possible to utilize the visible light rays as illumination light.

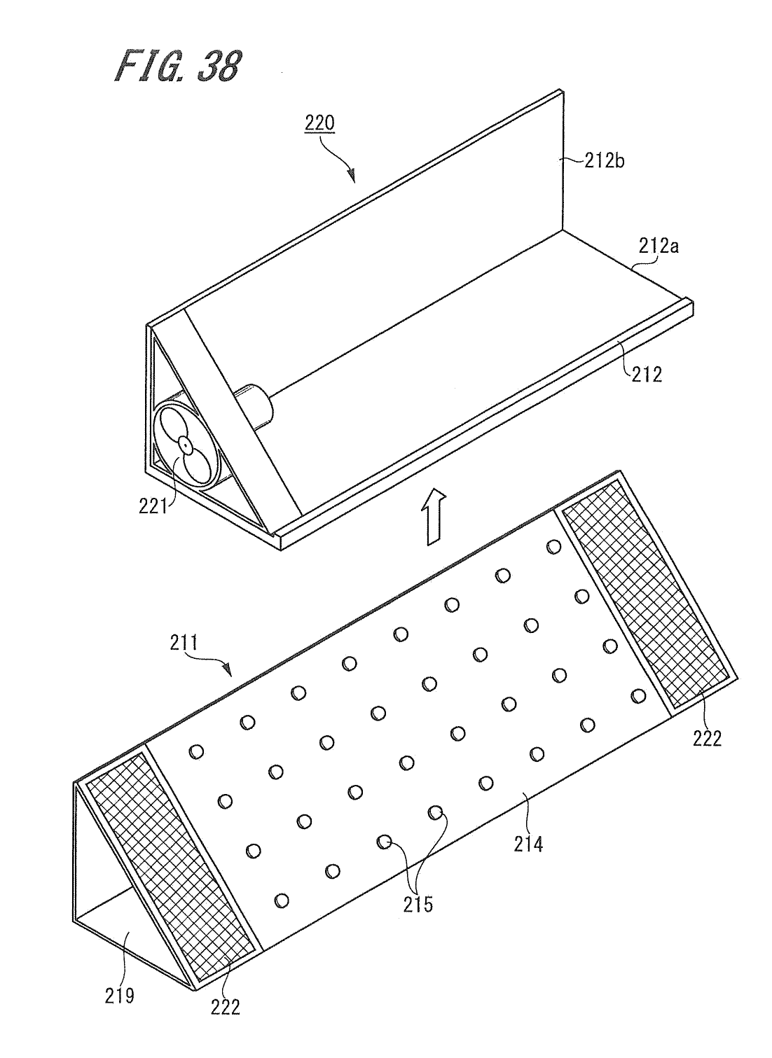

FIG. 38 is a diagram showing the first variation of the tenth embodiment according to the lighting and air cleaning device of the present invention. A lighting and air cleaning device 220 of the first variation of the tenth embodiment differs from the lighting and air cleaning device 210 described above in that a fan 221 and solar panels 222, 222 are provided. Hence, here, the fan 221 and the solar panels 222 will be described, like parts such as the light emission member stand 212, the substrate 214, etc. are identified with like symbols, and overlapped description will not be repeated.

The fan 221 is provided to forcibly send and move air into the air passage 217 formed of the light emission member stand 212 and the substrate 214. The solar panels 222 produce electrical energy for driving the fan 221. The electrical energy produced by the solar panels 222 may be directly used for driving the fan 221, or may be temporarily stored in a storage battery or the like and be taken and used as necessary. The solar panels 222 can produce electrical energy based on electromagnetic waves emitted from another lighting device, the sun or the like.

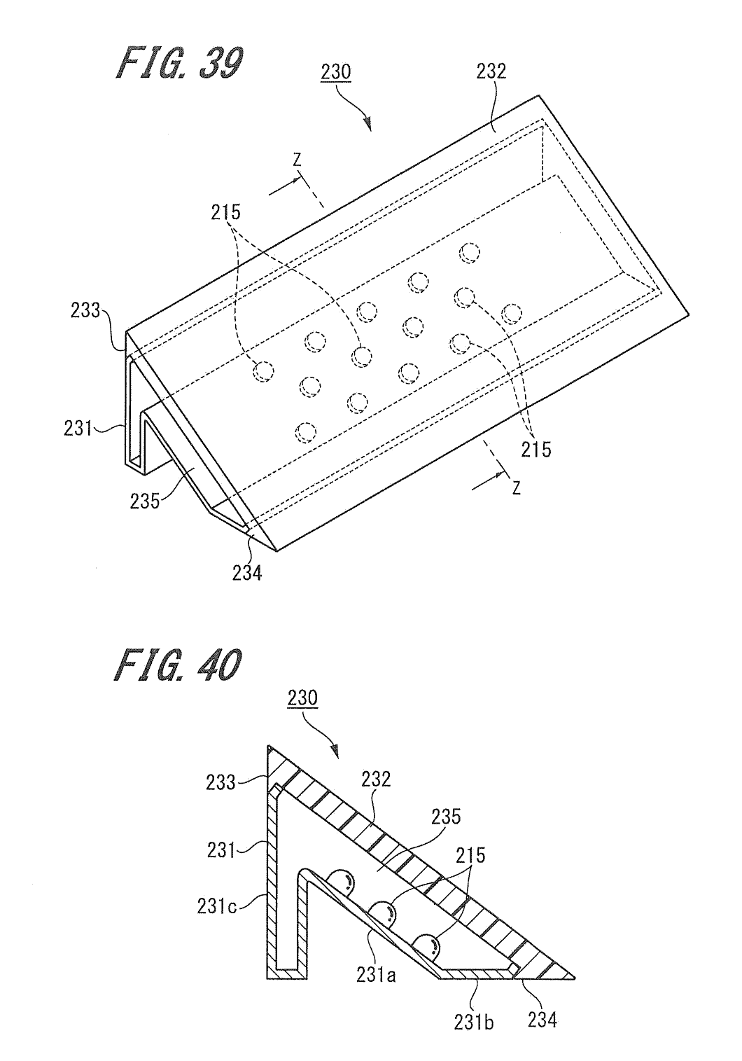

FIGS. 39 and 40 are diagrams showing the second variation of the tenth embodiment according to the lighting and air cleaning device of the present invention. The lighting and air cleaning device 230 of the second variation of the tenth embodiment differs from the lighting and air cleaning device 210 described above in a light emission member stand 231 and a resin cover 232. Hence, here, the light emission member stand 231 and the resin cover 232 will be described, like parts such as the ultraviolet generation light emitting diodes 215, etc. are identified with like symbols, and overlapped description will not be repeated.

The light emission member stand 231 includes a substrate portion 231a on which a plurality of ultraviolet generation light emitting diodes 215 are mounted, a bottom surface portion 231b which fixes and supports the substrate portion 231a at a predetermined inclination angle, and a side surface portion 231c which continuously rises on the back surface side of the bottom surface portion 231b. The resin cover 232 is removably mounted to the upper surface of the light emission member stand 231. As the material of the resin cover 232, the ultraviolet transmissive and visible-light transmissive fluorine resin is used, and the resin cover 232 is formed such that its thickness is set at about 5 mm. As described above, the ultraviolet transmissive and visible-light transmissive fluorine resin is used as the material, and the thickness is set at about 5 mm, and thus it is possible to absorb ultraviolet rays by 100% and prevent ultraviolet rays emitted by the ultraviolet generation light emitting diodes 215 set on the inside from being emitted to the outside. The resin cover 232 can also be formed by setting its thickness at about 1.5 mm to 3.0 mm, using the ultraviolet transmissive and visible-light transmissive fluorine resin material, and by coating at least one of its inner and outer surfaces with a fluorescent substance and the surface thereof with the ultraviolet transmissive and visible-light transmissive fluorine resin.

In the upper end edge of the resin cover 232, an upper nail portion 233 that is engaged with the upper end edge of the light emission member stand 231 is provided, and in the lower end edge of the resin cover 232, a lower nail portion 234 that is engaged with the lower end edge of the light emission member stand 231 is provided. A space portion set between the resin cover 232 and the light emission member stand 231 forms an air passage 235. The light emission member stand 231 can be configured as an ultraviolet lamp stand by replacing the ultraviolet generation light emitting diodes 215 with an ultraviolet lamp.

With the lighting and air cleaning device 230 configured as described above also, it is possible to obtain the same effects as the lighting and air cleaning device 210 described previously or the like. Specifically, when the ultraviolet generation light emitting diodes 215 of the lighting and air cleaning device 230 are energized, ultraviolet rays emitted from the ultraviolet generation light emitting diodes 215 mounted on the substrate portion 231a into the interior of the air passage 235 directly act on bacteria, viruses, and the like floating in the air within the air passage 235, and thus it is possible to kill or sterilize the bacteria and the like with the ultraviolet rays. In this way, it is possible to kill or sterilize bacteria, viruses, and the like floating in the air passing through the air passage 235, and to clean the air within a building, and thus sterilization effects by ultraviolet rays can be effectively exerted.

In this case, although the ultraviolet generation light emitting diodes 215 mounted on the substrate portion 231a emit ultraviolet rays toward a human, the ultraviolet rays are absorbed by the resin cover 232 arranged in front of the ultraviolet generation light emitting diodes 215, and thus the ultraviolet rays are prevented from being irradiated to the eyes, the skin or the like of the human. On the other hand, the visible light rays are not absorbed by the resin cover 232 but pass through the resin cover 232, and illuminate the interior of the building to brightly illuminate the surrounding area. Hence, even if the visible light rays directly enter the eyes of the human, the human is not affected by the visible light rays. Moreover, even if the visible light rays are irradiated to skin, there is no possibility that the skin is affected. Hence, the lighting and air cleaning device 230 can exert the illumination functions as an illumination unit. The lighting and air cleaning device of the present invention is suitable for, for example, a refrigerator, a cupboard, a shoe box, a drawer, a closet, a toilet and a bathroom. Furthermore, the lighting and air cleaning device can also be utilized by being installed in a vehicle or a specific spot.

### INDUSTRIAL APPLICABILITY

Although the present invention has been described above, the present invention is not limited to the embodiments described above. For example, in the embodiments described above, as the specific example of the electromagnetic wave generation source, the ultraviolet lamp is applied, however, any light source can be used as long as it can generate light containing blue visible light rays in the range of wavelengths of ultraviolet rays or close to ultraviolet rays. For example, it is also possible to use a fluorescent tube, a high-pressure mercury tube, a low-pressure mercury tube, an ultra high-pressure mercury tube, an ultra low-pressure mercury tube, a xenon lamp or the like. As described above, many modifications are possible without departing from the spirit of the present invention.

### EXPLANATION OF SYMBOLS

1, 40, 50, 60, 70, 80, 90, 100, 110, 120, 130, 140, 150, 170, 190, 200, 210, 220, 230: lighting and air cleaning device, 2: electromagnetic wave generation source, 3: ultraviolet-shielding resin member, 4: hanging unit, 5, 171: ultraviolet lamp (electromagnetic wave generation source), 6, 173: glass tube of ultraviolet lamp, 7: ferrule, 8: ferrule pin, 11: casing, 13a, 13b: ultraviolet-shielding plate, 15a, 15b: holding plate, 27, 31: hanging hardware, 28, 157: socket, 33: building, 33a, 155: ceiling, 41: cover and casing (ultraviolet-shielding resin member), 51: cover enclosure (ultraviolet-shielding resin member), 53: attachment hook, 61, 81, 91: cover tube (ultraviolet-shielding resin member), 62: ferrule adaptor, 63: attachment ring (attachment member), 68, 94, 102, 164, 176, 204, 235: air passage, 69, 95, 177, 206, 207, 219: exit and entrance port, 71, 71A: cover cylinder (ultraviolet-shielding resin member), 72: attachment block (attachment member), 81, 91, 131: cover tube member (ultraviolet-shielding resin member), 82: support ring, 92: attachment ring (attachment member), 93: cover support bar, 101, 141: gutter-like cover member (ultraviolet-shielding resin member), 111, 121, 191, 211: diode light emission member (electromagnetic wave generation source), 114, 193, 215: ultraviolet generation light emitting diode, 122: visible light ray generation light emitting diode, 142: fixing arm, 151: attachment frame, 152: external plate, 162, 221: fan, 163, 222: solar panel, 172: one-end-opened cup (ultraviolet-shielding resin member), 174: ferrule member, 201: both-end-opened cup (ultraviolet-shielding resin member), 202: lid member, 203: hanging hook, 212, 231: light emission member stand, 213: tunnel tube member (ultraviolet-shielding resin member), 218: reflective plate, 232: resin cover.

1. A lighting and air cleaning device comprising:
an electromagnetic wave generation source that emits an electromagnetic wave including an ultraviolet ray and a visible light ray; and
an ultraviolet-shielding resin member that covers at least a part of an electromagnetic wave emission portion emitting the electromagnetic wave of the electromagnetic wave generation source so as to prevent the ultraviolet ray included in the electromagnetic wave from being directly irradiated to a human being,
wherein the ultraviolet-shielding resin member includes an ultraviolet non-transmissive and visible-light transmissive portion that blocks or absorbs the ultraviolet ray emitted from the electromagnetic wave generation source and that transmits the visible light ray emitted from the electromagnetic wave generation source,
wherein an air passage in which air around the electromagnetic wave emission portion can move is provided between the electromagnetic wave generation source and the ultraviolet-shielding resin member, and
wherein the ultraviolet ray emitted from the electromagnetic wave generation source is irradiated to the air moving in the air passage so as to kill or sterilize bacteria, viruses, and the like in the air, and the visible light ray emitted from the electromagnetic wave generation source and passing through the ultraviolet non-transmissive and visible-light transmissive portion is used as illumination light.

2. The lighting and air cleaning device according to claim 1, wherein
the ultraviolet non-transmissive and visible-light transmissive portion is formed of an ultraviolet non-transmissive and visible-light transmissive fluorine resin that blocks or absorbs an ultraviolet ray and that transmits a visible light ray, or
the ultraviolet non-transmissive and visible-light transmissive portion is formed by any of: kneading a light-emitting material that emits visible light rays by irradiation of an ultraviolet ray into an ultraviolet transmissive and visible-light transmissive fluorine resin that transmits an ultraviolet ray and a visible light ray; applying a light-emitting material that emits visible light rays by irradiation of an ultraviolet ray, to a surface of an ultraviolet transmissive and visible-light transmissive fluorine resin that transmits an ultraviolet ray and a visible light ray, on a side opposite to the electromagnetic wave generation source; and applying a light-emitting material that emits visible light rays by irradiation of an ultraviolet ray, to at least a surface of a resin that transmits an ultraviolet ray and a visible light ray or of an ultraviolet transmissive and visible-light transmissive fluorine resin, on a side of the electromagnetic wave generation source.

3. The lighting and air cleaning device according to claim 1,
wherein the ultraviolet non-transmissive and visible-light transmissive portion includes:
a resin base sheet formed of an ultraviolet non-transmissive and visible-light transmissive resin plate or an ultraviolet non-transmissive and visible-light transmissive resin film that blocks or absorbs an ultraviolet ray and that transmits a visible light ray; and
a coated sheet that is provided by coating on both surfaces of the base sheet and that is made of ultraviolet transmissive and visible-light transmissive fluorine resin which transmits an ultraviolet ray and a visible light ray.

4. The lighting and air cleaning device according to claim 1,
wherein the ultraviolet-shielding resin member includes:
a casing that has an opening portion forming a part of the air passage and that has a space portion in which the electromagnetic wave generation source is housed; and
a hanging unit that hangs and supports the casing.

5. The lighting and air cleaning device according to claim 1,
wherein the ultraviolet-shielding resin member includes:
a casing that has an opening portion forming a part of the air passage, in which an entire portion other than the opening portion is formed as the ultraviolet non-transmissive and visible-light transmissive portion; and
a holding unit that is fixed to the casing and that removably holds the electromagnetic wave generation source, and
wherein the casing is fixed to a predetermined position.

6. The lighting and air cleaning device according to claim 1,
wherein the ultraviolet-shielding resin member includes:
a casing that has an opening portion forming a part of the air passage, in which an entire portion other than the opening portion is formed as the ultraviolet non-transmissive and visible-light transmissive portion; and
an engagement unit that is fixed to the casing and that can engage with the electromagnetic wave generation source, and
wherein the engagement unit is engaged with the electromagnetic wave generation source which is remobably held by a socket fixed to a predetermined position, and thereby the casing covers the electromagnetic wave emission portion.

7. The lighting and air cleaning device according to claim 1,
wherein the ultraviolet-shielding resin member includes:
a tubular ultraviolet non-transmissive and visible-light transmissive resin tube that covers at least a part of the electromagnetic wave emission portion; and
an attachment unit that attaches the ultraviolet non-transmissive and visible-light transmissive resin tube to the electromagnetic wave generation source, and
wherein the attachment unit is attached to the electromagnetic wave generation source, and the ultraviolet non-transmissive and visible-light transmissive resin tube is screwed to the attachment unit with an attachment screw.

8. The lighting and air cleaning device according to claim 7,
wherein the ultraviolet non-transmissive and visible-light transmissive resin tube includes one ultraviolet non-transmissive and visible-light transmissive resin plate or ultraviolet non-transmissive and visible-light transmissive resin film having:
a resin base sheet that blocks or absorbs an ultraviolet ray and that transmits a visible light ray; and a coated sheet that is integrally provided on both surfaces of the base sheet by coating and that is made of ultraviolet transmissive and visible-light transmissive fluorine resin which transmits an ultraviolet ray and a visible light ray, and
wherein the ultraviolet non-transmissive and visible-light transmissive resin tube is formed into a tubular shape by inflecting the ultraviolet non-transmissive and visible-light transmissive resin plate or the ultraviolet non-transmissive and visible-light transmissive resin film to form a circular or elliptical cross-sectional shape or by bending the plate or film at a plurality of places to form a polygonal cross-sectional shape.

9. The lighting and air cleaning device according to claim 1,
wherein the ultraviolet-shielding resin member includes:
a tubular ultraviolet non-transmissive and visible-light transmissive resin tube that covers the electromagnetic wave emission portion; and
a support unit that supports the ultraviolet non-transmissive and visible-light transmissive resin tube to the electromagnetic wave generation source, and
wherein the support unit is attached to the electromagnetic wave generation source and the ultraviolet non-transmissive and visible-light transmissive resin tube is supported to the support unit, and thereby the air passage is formed between the ultraviolet non-transmissive and visible-light transmissive resin tube and the electromagnetic wave generation source.

10. The lighting and air cleaning device according to claim 1,
wherein the ultraviolet-shielding resin member includes one ultraviolet non-transmissive and visible-light transmissive resin plate or ultraviolet non-transmissive and visible-light transmissive resin film having:
a resin base sheet that blocks or absorbs an ultraviolet ray and that transmits a visible light ray; and a coated sheet that is integrally provided on both surfaces of the base sheet by coating and that is made of ultraviolet transmissive and visible-light transmissive fluorine resin which transmits an ultraviolet ray and a visible light ray,
wherein the ultraviolet non-transmissive and visible-light transmissive resin plate or the ultraviolet non-transmissive and visible-light transmissive resin film is formed into a U-shaped or V-shaped cross-sectional shape to provide an ultraviolet non-transmissive and visible-light transmissive resin gutter, and the ultraviolet non-transmissive and visible-light transmissive resin gutter covers at least a part of the electromagnetic wave emission portion,
wherein an attachment unit is provided that attaches the ultraviolet non-transmissive and visible-light transmissive resin gutter to the electromagnetic wave generation source, and
wherein the ultraviolet non-transmissive and visible-light transmissive resin gutter is screwed to the attachment unit with an attachment screw.

11. The lighting and air cleaning device according to claim 10,
wherein the attachment unit includes an attachment arm to which the ultraviolet-shielding resin member is fixed, and
wherein the ultraviolet-shielding resin member is screwed to the attachment arm with an attachment screw.

12. The lighting and air cleaning device according to claim 1,
wherein the electromagnetic wave emission portion of the electromagnetic wave generation source is in a shape of a bar that is linearly extended, and
wherein the ultraviolet-shielding resin member includes:
a pair of attachment rings that is mounted to both sides of the electromagnetic wave emission portion in a longitudinal direction;
a plurality of support bars that is placed between the pair of attachment rings; and
an ultraviolet non-transmissive and visible-light transmissive resin tube that is supported by the plurality of support bars to form the air passage and that blocks or absorbs an ultraviolet ray and transmits a visible light ray.

13. The lighting and air cleaning device according to claim 1,
wherein the electromagnetic wave generation source has a ferrule which is integral with the electromagnetic wave emission portion and in which a ferrule pin is provided,
wherein the lighting and air cleaning device further comprises a ferrule adaptor to which the ferrule is removably mounted and which can be electrically connected to the ferrule pin, the ferrule adapter having a connection pin that is connected to a connection terminal of a socket connected to a power source, and
wherein the electromagnetic wave generation source can be connected to the socket through the ferrule adaptor.

14. The lighting and air cleaning device according to claim 1,
wherein the ultraviolet-shielding resin member includes:
an external plate in which an opening window for irradiating a visible light ray is provided;
an ultraviolet non-transmissive and visible-light transmissive portion that covers the opening window and that is formed of an ultraviolet non-transmissive and visible-light transmissive resin plate or an ultraviolet non-transmissive and visible-light transmissive resin film that blocks or absorbs an ultraviolet ray and that transmits a visible light ray; and
an attachment plate to which the external plate is fixed,
wherein a socket to which the electromagnetic wave generation source is removably mounted is attached to the attachment plate, and a surface of the attachment plate facing the electromagnetic wave emission portion is formed as a reflective surface, and
wherein the air passage is provided between a surface of the ultraviolet non-transmissive and visible-light transmissive portion of the ultraviolet-shielding resin member and the attachment plate.

15. The lighting and air cleaning device according to claim 1,
wherein the electromagnetic wave generation source includes a ferrule member in which the electromagnetic wave emission portion protrudes from one surface thereof,
wherein the ultraviolet-shielding resin member has a tubular cover body having a bottom, in which the ferrule member is fitted to an opening portion provided on one side, in which a notch forming a part of the air passage is provided, and in which one end is closed, and
wherein the cover body covers the entire electromagnetic wave emission portion, and is formed of a substance that blocks or absorbs an ultraviolet ray and that transmits a visible light ray.

16. The lighting and air cleaning device according to claim 1,
wherein the electromagnetic wave generation source includes:
an electromagnetic wave emission portion that is formed into a U-shape or V-shape having a bent portion; and
a ferrule member in which the electromagnetic wave emission portion protrudes from one surface thereof,
wherein the ultraviolet-shielding resin member includes:
a tubular cover body in which the ferrule member is fitted to a first opening portion provided on one end, in which a notch forming a part of the air passage is provided and which has opening portions on both ends, and
a cover lid member that is provided so as to close the first opening portion of the cover body and a second opening portion on an opposite side,
wherein the cover body covers the entire electromagnetic wave emission portion, and is formed of a substance that blocks or absorbs an ultraviolet ray and that transmits a visible light ray, and
wherein a hook member is provided in the cover lid member, and the hook member is hooked on the bent portion of the electromagnetic wave emission portion, and thereby the cover lid member closes the second opening portion of the cover body.

17. The lighting and air cleaning device according to claim 1,
wherein the electromagnetic wave generation source has an ultraviolet lamp that generates an electromagnetic wave in a wavelength range of at least 180 to 379 nm.

18. The lighting and air cleaning device according to claim 1,
wherein the electromagnetic wave generation source has a light emitting diode that generates an electromagnetic wave in a wavelength range of at least 180 to 379 nm.

19. The lighting and air cleaning device according to claim 1,
wherein the electromagnetic wave emission portion of the electromagnetic wave generation source includes a plurality of light emitting diodes that generates an electromagnetic wave in a wavelength range of at least 180 to 379 nm on one surface of a wiring substrate,
wherein the lighting and air cleaning device further comprises a stand member in which the electromagnetic wave emission portion is disposed inside and against which the wiring substrate is rested, and
wherein the ultraviolet-shielding resin member is formed of a curtain member that covers a side of the electromagnetic wave emission portion of the electromagnetic wave generation source which is propped against the stand member.

20. The lighting and air cleaning device according to claim 19,
wherein a plurality of light emitting diodes for visible light that generate an electromagnetic wave in a wavelength range of 380 to 780 nm is provided on a surface opposite to the surface of the wiring substrate on which the light emitting diodes that generate an electromagnetic wave in a wavelength range of at least 180 to 379 nm are provided.

21. The lighting and air cleaning device according to claim 1,
wherein the electromagnetic wave generation source has a semiconductor light source that generates an electromagnetic wave in a wavelength range of at least 180 to 379 nm by irradiating an electron beam to a semiconductor.

22. The lighting and air cleaning device according to claim 1,
wherein a fan that forcibly circulates air is provided within the air passage.
